SEQUENCE LISTING (2 sequences)

SEQ ID NO: 1 - DNA, 24 bp, Artificial Sequence
  Description of Artificial Sequence  Synthetic Primer
gatccttctgggaattcctagatc

SEQ ID NO: 2 - DNA, 22 bp, Artificial Sequence
  Description of Artificial Sequence  Synthetic Primer
agttgaggggactttcccaggc

(19) United States
(12) Patent Application Publication
(10) Pub. No.: US 2005/0143336 A1
(43) Pub. Date: Jun. 30, 2005

(54) Methods and compositions for improved non-viral gene therapy

(75) Inventors: Rajagopal Ramesh, Sugar Land, TX (US); Began Gopalan, Houston, TX (US); Jack A. Roth, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 11/000,341

(22) Filed: Nov. 30, 2004

Related U.S. Application Data
(60) Provisional application No. 60/533,180, filed on Dec. 30, 2003.

(51) Int. Cl. A61K 48/00; A61K 38/13; A61K 31/70; A61K 31/60; A61K 31/525

(52) U.S. Cl. 514/44; 514/159; 514/400; 514/406; 514/420; 514/569; 514/570; 514/165; 514/290; 514/492; 514/23; 514/11; 514/251; 514/449

(57) ABSTRACT

Methods to prevent or reduce inflammation secondary to administration of a lipid-nucleic acid complex in a subject, that include administering to the subject a non-steroidal anti-inflammatory agent, a salicylate, an anti-rheumatic agent, an antihistamine, or an immunsuppressive agent with the lipid-nucleic acid complex are disclosed. Also disclosed are methods of screening for inhibitors of the inflammatory response associated with administration of a lipid-nucleic acid complex to a subject, including providing a candidate substance suspected of preventing or inhibiting the inflammation associated with administration of a lipid-nucleic acid complex to the subject. Also disclosed are compositions that include a lipid, a nucleic acid, and a non-steroidal anti-inflammatory agent, a salicylate, an anti-rheumatic agent, an antihistamine, or an immunosuppressive agent.

This application claims the benefit of the filing date of U.S. provisional patent application Ser. No. 60/533,180, filed Dec. 30, 2003, the entire contents of which is hereby incorporated by reference.

### BACKGROUND OF THE INVENTION

1. Field of the Invention

The present invention relates generally to the fields of molecular biology and pharmacology. More particularly, it concerns methods to prevent or reduce inflammation secondary to administration of a lipid-nucleic acid complex in a subject, involving administering to the subject a non-steroidal anti-inflammatory agent, a salicylate, an anti-rheumatic agent, an antihistamine, or an immunosuppressive agent with the lipid-nucleic acid complex. The present invention also concerns methods of screening for inhibitors of the inflammatory response association with administration of a lipid-nucleic acid complex to a subject. In addition, the present invention concerns compositions that include a lipid, a nucleic acid, and a non-steroidal anti-inflammatory agent, a salicylate, an anti-rheumatic agent, an antihistamine, or an immunosuppressive agent.

2. Description of Related Art

Gene therapy involves the expression of foreign genes in cells with the intention of providing a therapeutic benefit. Clinical trials involving treatment of cancer have developed into one of the most important indications for gene therapy.

At present, viral and nonviral methods of gene transfer are used both in vivo and ex vivo/in vitro. Viral vectors currently used in clinical trials include retroviruses, adenoviruses, adeno-associated viruses, and herpes viruses. However, viral vectors are limited by (1) their relatively small capacity for therapeutic DNA, (2) safety concerns, and (3) difficulty in targeting to specific cell types. These difficulties have led to the evaluation and development of alternative vectors based on synthetic, non-viral systems.

One of the main alternatives to viral vectors is the liposome-DNA complex. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

The size of a liposome varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Negatively charged, or classical, liposomes have been used to deliver encapsulated drugs for some time and have also been used as vehicles for gene transfer into cells in culture. Problems with the efficiency of nucleic acid encapsulation, coupled with a requirement to separate the DNA-liposome complexes from “ghost” vesicles, has led to the development of positively-charged liposomes.

Cationic liposomes can be formed from various cationic lipids. Examples of these lipids include DOTAP (N-1(2,3-dioleoyloxy)propyl)-N,N,N-trimethylammoniumethyl sulphate) and DOTMA (N-(1-(2,3-dioleoyloxy)propyl)-N,N,N-trimethylammonium chloride). Cationic liposomes often incorporate a neutral lipid, such as DOPE (dioleoylphosphatidylethanolamine), into the formation to facilitate membrane fusion.

The efficient transfection of eukaryotic cells using cationic liposomes was first described in 1987 by Felgner et al. Cationic liposomes were shown to bind DNA efficiently, leading to cellular uptake of plasmid DNA and a high level of transgene expression. Cationic liposomes are able to interact spontaneously with negatively charged DNA to form clusters of aggregated vesicles along the nucleic acid. At a critical density, the DNA is condensed and becomes encapsulated within a lipid bilayer. There is some evidence that cationic liposomes do not actually encapsulate the DNA, but instead bind along the surface of the DNA, maintaining its original size and shape.

Liposomes interact with cells to deliver agents via four different mechanisms: (1) endocytosis by phagocytic cells of the reticuloendothelial system such as macrophages and/or neutrophils; (2) adsorption to the cell surface, either by nonspecific weak hydrophobic and/or electrostatic forces, and/or by specific interactions with cell-surface components; (3) fusion with the plasma cell membrane by insertion of the lipid bilayer of the liposome into the plasma membrane, with simultaneous release of liposomal contents into the cytoplasm; (4) by transfer of liposomal lipids to cellular and/or subcellular membranes, and/or vice versa, without any association of the liposome contents. Varying the liposome formulation can alter which mechanism is operative, although more than one may operate at the same time.

Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively-charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

It has been shown that the uptake of lipid-DNA complex particles by immune cells triggers a strong inflammatory response (Dow et al., 1999; Li et al., 1999; Tousignant et al., 2000). Vector administration has been found to induce a potent inflammatory response in mice, characterized by complement activation and the induction of the cytokines IFN-γ, TNF-α, IL-6, and IL-12 (Tousignant et al., 2000). These toxicities were found to be transient, and were independent of the lipid:DNA ratio, the cationic lipid species, and the level of transgene expression attained (Tousignant et al., 2000). It has also been reported that cationic lipid-DNA complexes injected intravenously produce marked immune activation, including upregulation of CD69 expression on multiple cell types and systemic release of high levels of Th1 cytokines, from both lung and spleen mononuclear cells (Dow et al., 1999).

It is believed that the inflammatory response associated with lipid-DNA complex administration is largely due to 5′-cytosine-guanosine-3′ (CpG) motifs in the plasmid DNA (Krieg et al., 1995; Klinman et al., 1996; Ballas et al., 1996; Sparwasser et al., 1998). It has been shown that bacterial DNA and immunostimulatory CpG oligodeoxynucleotides activate macrophages in vivo and in vitro to express activation markers, to translocate NF-κB, and to secrete pro-inflammatory cytokines such as TNF-α, IL-1, IL-6, and IL-12 (Sparwasser et al., 1997; Lipford et al., 1997; Stacey et al., 1996).

While the induction of potentially useful cytokines such as IL-12 or potentially harmful cytokines such as TNF-α appears to be dependent on the sequence of the CpG-oligodeoxynucleotides used, a hallmark of bacterial DNA and immunostimulatory CpG-oligodeoxynucleotids is its profound adjuvanticity for the induction of murine Th1 responses (Lipford et al., 1997; Roman et al., 1997; Chu et al., 1997). Bacterial DNA and immunostimulatory CpG-oligodeoxynucleotides also cause maturation and activation of dendritic cells to bring about conversion of immature dendritic cells into mature antigen-presenting cells (Sparwasser et al., 1998). The level of proinflammatory cytokines can become very high at the vector dose that achieves significant transgene expression (Dow et al., 1999; Li et al., 1999; Tousignant et al., 2000). Among the cytokines, TNF-α is believed to be the primary source of toxicity (Tan et al., 2002). At high concentrations, it induces septic shock in animals as well as inhibiting expression of the transgene (Li et al., 1999; Tan et al., 1999). Recently, NF-κB has been suggested to be a target for anti-inflammatory therapy (Tan et al., 2002).

In view of the above, strategies to prevent or reduce the inflammation associated with administration of lipid-DNA complexes in a subject will not only assist in lessening the toxicity of this form of gene therapy, but will also improve the transgene expression associated with administration of lipid-DNA complexes. Measures to decrease the inflammation associated with lipid-DNA complexes will help to make gene transfer using lipid-DNA complexes a more viable option in the treatment of diseases such as cancer.

### SUMMARY OF THE INVENTION

The inventors have discovered that anti-inflammatory drugs provide protection against the toxicity associated with administration of lipid-nucleic acid complexes. The inventors discovered that this protection is the result of downregulation of NF-κB, a potent stimulator of inflammation. These findings indicate that anti-inflammatory drugs (such as non-steroidal anti-inflammatory agents, salicylates, anti-rheumatic agents, antihistamines, immunosuppressive agents, and related agents) can protect against the toxicity associated with administration of lipid-nucleic acid complexes.

Certain embodiments of the present invention are generally concerned with methods to prevent or reduce inflammation secondary to administration of a lipid-nucleic acid complex in a subject, comprising administering to the subject an agent with the lipid-nucleic acid complex, wherein the agent is selected from the group consisting of a non-steroidal anti-inflammatory agent, a salicylate, an anti-rheumatic agent, an antihistamine, and an immunosuppressive agent. Inflammation secondary to any lipid-nucleic acid complex in a subject is contemplated by the present methods.

Any mechanism of inflammation secondary to administration of a lipid-nucleic acid complex may be prevented or reduced by the present invention. In certain embodiments of the present invention, the nucleic acid contains CpG sites that induce inflammation. In other embodiments, the inflammation is secondary to upregulation of NFκB in the subject.

Any method of administering to the subject a non-steroidal anti-inflammatory agent, a salicylate, an anti-rheumatic agent, an antihistamine, or an immunosuppressive agent with the lipid-nucleic acid complex is contemplated by the present invention. In certain embodiments, the non-steroidal anti-inflammatory agent, the salicylate, the anti-rheumatic agent, an antihistamine, or the immunosuppressive agent is administered to the subject concurrently with the lipid-nucleic acid complex. For example, the non-steroidal anti-inflammatory agent, the salicylate, the anti-rheumatic agent, the antihistamine, or the immunosuppressive agent may be incorporated into the lipid-nucleic acid complex. In certain other embodiments, the non-steroidal anti-inflammatory agent, the salicylate, the anti-rheumatic agent, the antihistamine, or the immunosuppressive agent is administered to the subject separately from the lipid-nucleic acid complex. In further embodiments, the non-steroidal anti-inflammatory agent, the salicylate, the anti-rheumatic agent, the antihistamine, or the immunosuppressive agent is administered to the subject prior to administration of the lipid-nucleic acid complex. Alternatively, the non-steroidal anti-inflammatory agent, the salicylate, the anti-rheumatic agent, the antihistamine, or the immunosuppressive agent may be administered to the subject following administration of the lipid-nucleic acid complex.

In certain embodiments, the methods of the present invention involve administering to the subject two or more agents selected from the group consisting of a non-steroidal anti-inflammatory agent, a salicylate, an anti-rheumatic agent, an antihistamine, and an immunosuppressive agent. For example, the methods may involve administering to the subject a non-steroidal anti-inflammatory agent and a salicylate, a salicylate and an antirheumatic agent, an antirheumatic agent and an immunosuppressive agent, a non-steroidal antiinflammatory agent and an immunosuppressive agent, a salicylate and an immunosuppressive agent, a non-steroidal anti-inflammatory agent and an antihistamine, a salicylate and an antihistamine, an anti-rheumatic agent and an antihistamine, an immunosuppressive agent and an antihistamine, or a non-steroidal anti-inflammatory agent and an anti-rheumatic agent.

Any non-steroidal anti-inflammatory agent, salicylate, anti-rheumatic agent, antihistamine, or immunosuppressive agent is contemplated by the methods of the present invention. One of ordinary skill in the art is familiar with the wide variety of these agents that are available.

Examples of non-steroidal anti-inflammatory agents include diflunisal, ibuprofen, fenoprofen, flurbiprofen, ketoprofen, nabumetone, piroxicam, naproxen, naproxen sodium, diclofenac, diclofenac sodium and misoprostol, indomethacin, sulindac, etodolac, tolmetin, etodolac, ketorolac, oxaprozin, rofecoxib, mefenamic acid, meclofenamate, celecoxib, and vioxx. In certain particular embodiments, the anti-inflammatory agent is naproxen. Examples of salicylates include acetylsalicylic acid, sodium salicylate, choline salicylate, choline magnesium salicylate, diflunisal, or salsalate, choline magnesium trisalicylate. Examples of anti-rheumatic agents include gold sodium thiomalate, aurotheioglucose, auranofin, chloroquine, hydroxychloroquine, penicillamine, leflunomide, etanercept, infliximab, azathioprine, or sulfasalazine. Examples of antihistamines include diphenhydramine, chlorpheniramine, clemastine, hydroxyzine, triprolidine, loratadine, cetirizine, fexofenadine, or desloratadine. Examples of immunosuppressive agents include cyclosporine A, azathoprine, methotrexate, mechorethamine, cyclophosphamide, chlorambucil, or mycophenolate mofetil. In certain embodiments of the present compositions, the immunosuppressive agent is cyclosporine A.

In certain other embodiments of the present invention, the non-steroidal anti-inflammatory agent is an inhibitor of an inflammation-associated signaling molecule, such as p38MAPK or p44/42MAPK. P38MAPK and p44/42MAPK are examples of inflammation-associated signaling molecules. As set forth in the examples below, small molecule inhibitors targeted to p38MAPK or p44/42MAPK or COX-2 have been demonstrated to suppress the inflammation associated with administration of lipid-nucleic acid complexes. Thus, in certain embodiments, for example, the inhibitor of p38MAPK is SB 203580. The inhibitor of p44/42MAPK may be any inhibitor of p44/42MAPK, such as U0126.

Any nucleic acid is contemplated for inclusion in the methods of the present invention, as long as the nucleic acid is capable of forming a lipid-nucleic acid complex. For example, the nucleic acid may be a deoxyribonucleic acid (DNA). In certain embodiments, the deoxyribonucleic acid may include a therapeutic gene. Any therapeutic gene known to those of ordinary skill in the art may be included in the DNA. Examples of classes of therapeutic genes include, for example, tumor suppressor genes, genes that induce apoptosis, genes encoding an enzyme, genes encoding an antibody, or genes encoding a hormone.

One of ordinary skill in the art would be familiar with the class of genes known as tumor suppressor genes and with genes that induce apoptosis. These classes of therapeutic genes have anti-cancer properties. Examples of such therapeutic genes include Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, FUS1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include the tumor suppressor genes at 3p21.3, including FUS1, Gene 26 (CACNA2D2), PL6, Beta*(BLU), LUCA-1 (HYAL1), LUCA-2 (HYAL2), 123F2 (RASSF1), 101F6, Gene 21 (NPRL2), and SEM A3. In certain particular embodiments, the therapeutic gene is FUS1.

As discussed above, the therapeutic gene may be a gene encoding an antibody, a hormone, or an enzyme. Examples of these genes are discussed in greater detail in the specification below.

In certain embodiments of the present invention, the DNA is antisense DNA. Any antisense DNA can be applied in the methods of the present invention. For example, the antisense DNA may be antisense ras, antisense myc, antisense raf, antisense erb, antisense src, antisense fms, antisense jun, antisense trk, antisense ret, antisense gsp, antisense hst, antisense bcl, or antisense abl.

In certain embodiments of the present invention, the nucleic acid is ribonucleic acid (RNA). For example, the RNA may be messenger RNA, antisense RNA, or interfering RNA. In other embodiments, the RNA further comprises a ribozyme. Alternatively, the nucleic acid may be a DNA-RNA hybrid.

The present invention contemplates any type of lipid for inclusion in the lipid-nucleic acid complexes of the present invention. One of ordinary skill in the art would be familiar with the many types of lipids that are known in the art. For example, in certain embodiments, the lipid is a cationic lipid, such as DOTAP or DOTMA. In other embodiments, the lipid is a neutral lipid, such as DOPE.

In other embodiments of the present invention, the lipid is included in a liposome. Any liposome is contemplated for inclusion in the present invention. One of ordinary skill in the art would be familiar with liposomes and the many types of liposomes that are available for inclusion in the present invention. For example, the liposome may be a unilamellar liposome or a multilamellar liposome. In certain embodiments, the lipid is comprised in a nanoparticle, or submicron particle. For example, the nanoparticle may have a diameter of from about 1 to about 100 nanometers.

In certain particular embodiments of the present invention, the lipid-nucleic acid complex comprises a lipid composition that includes DOTAP, cholesterol, the nucleic acid includes FUS1, and the non-steroidal anti-inflammatory agent is naproxen. In other particular embodiments, the lipid-nucleic acid complex comprises a lipid composition that includes DOTAP, cholesterol, the nucleic acid includes FUS1, and the non-steroidal anti-inflammatory agent is cyclosporine A. Any ratio of DOTAP and cholesterol is contemplated in these embodiments of the present invention. Furthermore, embodiments of the invention may include a nucleic acid that includes more than one therapeutic gene, such as FUS1 and another therapeutic gene. In addition, the composition may or may not include additional anti-inflammatory agents.

The present invention also pertains to methods of screening for inhibitors of the inflammatory response associated with administration of a lipid-nucleic acid complex to a subject, including: (1) providing a candidate substance suspected of preventing or inhibiting the inflammation associated with administration of a lipid-nucleic acid complex; (2) contacting a composition that includes the lipid-nucleic acid complex and the candidate substance with the subject, and (3) assaying for inflammation in the subject.

Any candidate substance suspected of preventing or inhibiting the inflammation associated with administration of a lipid-nucleic acid complex is contemplated for inclusion in the present invention. For example, the inhibitor of inflammation may be a small molecule, a peptide, a polypeptide, a protein, an oligonucleotide, a polynucleotide, or an antibody.

Any type of subject can be used in the screening methods of the present invention. For example, in certain embodiments, the subject is a human. The human may or may not be affected by a disease process. For example, in certain embodiments, the human is a patient with a hyperproliferative disease, such as cancer. Any type of nucleic acid is contemplated for inclusion in the screening methods of the present invention. For example, the nucleic acid may be a deoxyribonucleic acid (DNA). In some embodiments, the deoxyribonucleic acid includes a therapeutic gene, such as a tumor suppressor gene, a gene that induce apoptosis, a gene encoding an enzyme, a gene encoding an antibody, or a gene encoding a hormone. These have been discussed above, and are addressed in greater detail in the specification below. For example, the therapeutic gene may be Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, FUS1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include the tumor suppressor genes at 3p21.3, including FUS1, Gene 26 (CACNA2D2), PL6, Beta*(BLU), LUCA-1 (HYAL1), LUCA-2 (HYAL2), 123F2 (RASSF1), 101F6, Gene 21 (NPRL2), and SEM A3.

In some embodiments, the DNA is antisense DNA, such as antisense ras, antisense myc, antisense raf, antisense erb, antisense src, antisense fms, antisense jun, antisense trk, antisense ret, antisense gsp, antisense hst, antisense bcl, or antisense abl. One of ordinary skill in the art would be familiar with antisense DNA, and other antisense DNA that may be included in the methods of the present invention. As with the previously described methods, the nucleic acid may be RNA, such as messenger RNA, antisense RNA, or interfering RNA. In some embodiments, the RNA further includes a ribozyme. In other embodiments, the nucleic acid is a DNA-RNA hybrid.

The lipid-nucleic acid complexes of the present screening methods may include any lipid known to those of ordinary skill in the art. For example, the lipid may a cationic lipid, such as DOTAP or DOTMA. In other embodiments of the present invention, the lipid is a neutral lipid, such as DOPE. The lipid may be included in a liposome. For example, the liposome may be a unilamellar or multilamellar liposome.

The present invention also pertains to compositions that include (1) a lipid; (2) a nucleic acid; and (3) a non-steroidal anti-inflammatory agent, a salicylate, an anti-rheumatic agent, an antihistamine, or an immunosuppressive agent.

In certain embodiments of the present invention, the composition further includes two or more agents selected from the group consisting of a non-steroidal anti-inflammatory agent, a salicylate, an anti-rheumatic agent, an antihistamine, and an immunosuppressive agent. For example, the composition may include a non-steroidal anti-inflammatory agent and a salicylate, a salicylate and an antirheumatic agent, an antirheumatic agent and an immunosuppressive agent, a non-steroidal antiinflammatory agent and an immunosuppressive agent, a salicylate and an immunosuppressive agent, a non-steroidal anti-inflammatory agent and an antihistamine, a salicylate and an antihistamine, an anti-rheumatic agent and an antihistamine, an immunosuppressive agent and an antihistamine, or a non-steroidal anti-inflammatory agent and an anti-rheumatic agent.

As discussed above, any non-steroidal anti-inflammatory agent, salicylate, anti-rheumatic agent, antihistamine, or immunosuppressive agent known to those of ordinary skill in the art may be included in the compositions of the present invention. Examples of each of these types of agents has been set forth above.

The compositions of the present invention may include any type of nucleic acid.

For example, in certain embodiments, the nucleic acid is DNA. The DNA may further include a therapeutic gene. Any therapeutic gene known to those of ordinary skill in the art may be included in the compositions of the present invention. Examples of such therapeutic agents are discussed above in relation to the methods of the present invention.

In other embodiments of the present compositions, the DNA is antisense DNA. For example, the DNA may be antisense ras, antisense myc, antisense raf, antisense erb, antisense src, antisense fms, antisense jun, antisense trk, antisense ret, antisense gsp, antisense hst, antisense bcl, or antisense abl. In other embodiments of the present compositions, the nucleic acid is RNA. For example, the RNA may be messenger RNA, antisense RNA, or interfering RNA. In some embodiments, the RNA further comprises a ribozyme. In further embodiments, the nucleic acid is a DNA-RNA hybrid.

Any lipid may be included in the compositions of the present invention. One of ordinary skill in the art would be familiar with the many types of lipids that may be included in the compositions of the present invention. In certain embodiments, the lipid is a cationic lipid, such as DOTAP or DOTMA. In other embodiments, the lipid is a neutral lipid, such as DOPE. The compositions of the present invention may include a lipid that is included in a liposome. One of ordinary skill in the art would be familiar with liposomes. For example, the liposome may be a unilamellar liposome or a multilamellar liposome. In certain embodiments, the lipid is comprised in a nanoparticle, or submicron particle. For example, the nanoparticle may have a diameter of from about 1 to about 100 nanometers.

In certain embodiments of the composition of the present invention, the composition includes a lipid composition that includes DOTAP and cholesterol, a nucleic acid that includes FUS1, and naproxen. In certain other embodiments of the present invention, the composition includes a lipid composition that includes DOTAP and cholesterol, a nucleic acid that includes FUS1, and cyclosporine A. Any ratio of DOTAP and cholesterol is contemplated in these embodiments of the present invention. In certain of these embodiments, the nucleic acid may include more than one therapeutic gene, such as FUS1 and an additional therapeutic genes, and more than one anti-inflammatory agent.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Analysis of Naproxen levels in the blood of mice following oral administration of Naproxen. Maximum detectable levels of Naproxen occurred between 3 to 4 hours following administration.

FIG. 2. Analyses of serum samples from mice that received DOTAP:Chol-FUS1 complex demonstrated proimflammatory cytokine production. Analysis of cytokine levels in animals that had received Naproxen prior to treatment with DOTAP:Chol-FUS1 complex demonstrated a 50% reduction in all of the cytokine levels.

FIG. 3. Animals that were treated with 5 mg/kg or 15 mg/kg Naproxen were protected from DOTAP:Chol-FUS1 DNA complex induced toxicity compared to animals that did not receive Naproxen. The protection offered by Naproxen was dose-dependent.

FIG. 4. Cyclosporin A protects mice from DOTAP:Chol-FUS1 complex induced toxicity in vivo.

FIG. 5. Cyclosporin A can inhibit DOTAP:Chol-FUS1 complex induced toxicity In vivo following oral administration of cyclosporin followed by intravenous DOTAP:Chol-FUS1 treatment.

FIG. 6. Inhibition of FUS1-nanoparticles induced PGE₂ production by naproxen. Cells were either not treated or treated with naproxen (0.5 mM) prior to transfection with FUS1-nanoparticles (2.5 μg DNA). Tissue culture supernatant was collected at various time points and analyzed for PGE₂ concentration using a PGE₂ enzyme immunoassay kit. A significantly inhibition in PGE₂ levels were observed in naproxen treated cells compared to cells that were not treated with naproxen. Naproxen inhibited PGE₂ levels at all time points tested. Data is represented as the average of triplicate wells. Bars denote standard error.

FIG. 7. Naproxen does not affect transgene expression. Cells were either not treated or treated with naproxen prior to transfection with luc-nanoparticles. At 2 h, 4 h, and 15 h after transfection cell lysates were prepared and assayed for luciferase activity. Luciferase activity was observed in both naproxen treated and untreated cells. However, a slight increase in luciferase activity was observed in naproxen treated cells. Luciferase activity was expressed as relative light units per milligram of protein (RLU/mg protein). Results are represented as the average triplicates. Error bar denotes standard error.

FIG. 8. FUS1-nanoparticles induced inflammatory response is inhibited by naproxen in vivo. Mice were divided into three groups and treated as follows: Group 1 received no treatment and served as control; Group 2 received an intravenous injection of FUS1-nanoparticles; Group 3 received an oral dose of naproxen (15 mg/Kg) 3 h prior to receiving an intravenous injection of FUS1-nanoparticles. Animals were euthanized at various time points and analyzed for TNF-α in the blood and signaling molecules in lung tissues. FUS1-nanoparticle-mediated TNF-α expression was markedly suppressed in Group 3 mice compared to TNF-α expression in Group 2 mice. Baseline TNF-α levels were observed in Group 1 mice. Bars denote standard error.

FIG. 9A-D. Effect of systemic delivery of increasing doses of FUS1:nanoparticle in C3H mice. Mice were injected intravenously with 100 μg of FUS1:nanoparticle, 4 mM nanoparticle, 100 μg of FUS1 and survival was monitored (A). Mice were administered intravenously with different concentration of FUS1:Nanoparticle and survival was monitored (B). Mice were injected intravenously with 100 μg of Nanoparticle:FUS1 complex, 4 mM Nanoparticle, 100 μg of FUS1 and blood was collected at different time points for the serum (C) and organ cytokine levels (D).

FIG. 10. Effect of FUS1:nanoparticle on inflammatory cytokines TNF-α, IL-6 and PGE₂ production and MAPK activation, STAT3 and COX-2 expression in RAW4.7 cells. RAW264.7 (1×10⁶ cells/well) were stimulated with medium, nanoparticle, FUS1 (2.5 μg/ml) and FUS1:nanoparticle (2.5 μg/ml) complex. At indicated time after transfection, culture supernatant was collected to measure TNF-α, IL-6 and PGE₂ production and cell lysates were subjected to 10% SDS-PAGE and then Western blots were performed using a specific Ab against the phosphorylated form of p38 (pp38), JNK (pJNKs), ERK (p44/42), STAT3 (pSTAT3 Ser727, pSTAT3 Tyr705) and COX-2. β-actin in each sample was used as the equal loading control.

FIG. 11A-D. Effect of Naproxen on protecting mice from FUS1:nanoparticle mediated toxicity in C3H mice. Mice were given orally two different doses of naproxen and two hrs later injected intravenously with 100 μg of FUS1:nanoparticle and monitored the survival of mice (A). Mice were administered orally 15 mg/kg Naproxen. At indicated times after oral delivery blood was collected at 2, 4, 6 and 15 h and analysed for naproxen concentration using HPLC (B). Mice were administered orally 15 mg/kg Naproxen and were injected intravenously with 100 μg of FUS1:nanoparticle. At indicated times after injection, blood and organ was collected at different time points. TNF-α, IL-6, IL-1a and IFN-γ levels in serum (C) and TNF-α, IL-6 (D) in organs were determined using specific immunoassay kit. Data represent means±SD.

FIG. 12. Naproxen inhibits the toxicity associated with nanoparticle:FUS1 complex. Mice were given orally 15 mg/kg of naproxen and two hrs later injected intravenously with 100 μg of FUS1:nanoparticle. At indicated times after injection, lungs, liver, spleen, kidney, intestine, ovary, heart were collected and stored in formalin for histopathologic analysis.

FIG. 13. Effect of naproxen on inflammatory cytokines TNF-α, IL-6 and PGE₂ production and MAPK activation, STAT3 and COX-2 expression induced by Nanoparticle:FUS1 complex in RAW4.7 cells. RAW264.7 (1×10⁶ cells/well) were incubated with 0.2% medium for 24 hrs and then treated with naproxen (0.5 mM) for 3-31/2 hrs and then transfected with FUS1:nanoparticle (2.5 μg/ml) complex. At indicated time after transfection, culture supernatant was collected to measure TNF-α, IL-6 and PGE₂ production and cell lysates were subjected to 10% SDS-PAGE and then Western blots were performed using a specific Ab against the phosphorylated form of p38 (pp38), JNK (pJNKs), ERK (p44/42), STAT3 (pSTAT3 Ser727, pSTAT3 Tyr705) and COX-2. β-actin in each sample was used as the equal loading control.

FIG. 14. Effect of naproxen on FUS1:nanoparticle mediated transfection of RAW264.7 cells. Cells were pretreated with naproxen and after 3 h 30 min, cells were transfected with nanoparticle:luciferase reporter plasmid pGL3CMV. Cells were harvested at 2, 4 and 15 h after transfection and cell lysates assayed for luciferase activity, normalized to protein content.

FIG. 15. Prolonged survival in C3H mice treated with p38MAPK inhibitor. Mice were received two doses of p38MAPK and JNK inhibitor intraperitonially at 24 h and 3 h before injecting FUS:nanoparticle complex intravenously. Mice were assessed for morbidity and mortality.

### DESCRIPTION OF ILLUSTRATIVE EMBODIMENTS

The inventors have discovered that anti-inflammatory drugs provide protection against the toxicity associated with administration of lipid-nucleic acid complexes. More particularly, the inventors have discovered that cyclosporine A and Naproxen protect mice from toxicity induced by DOTAP:cholesterol (Chol)-FUS1 DNA complex.

Intravenous administration of DOTAP:Chol-FUS1 complex was found to be lethal to animals, resulting in death of 100% of animals tested. However, oral administration of Cyclosporine A (100 mg/kg) completely protected the mice from death. The inventors discovered that this protection is the result of downregulation of NF-κB, a potent stimulator of inflammation. Further, the inventors have discovered that treatment of immunocompetent mice with Naproxen prior to treatment with DOTAP:Chol-FUS1 complex resulted in protection and survival of mice compared to animals that did not receive Naproxen.

These findings indicate that anti-inflammatory drugs (such as non-steroidal anti-inflammatory agents, salicylates, anti-rheumatic agents, antihistamines, immunosuppressive agents, and related agents) can protect against the toxicity associated with administration of lipid-nucleic acid complexes.

A. Nucleic Acids, Vectors, and Regulatory Signals

The present invention concerns methods to prevent or reduce inflammation secondary to administration of a lipid-nucleic acid complex in a subject. Additionally, the present invention is concerned with methods of screening for inhibitors of the inflammatory response associated with administration of a lipid-nucleic acid complex to a subject. The present invention is also concerned with compositions comprising a lipid, a nucleic acid, and a non-steroidal anti-inflammatory agent, a salicylate, an anti-rheumatic agent, an antihistamine, or an immunosuppressive agent.

1. Definition of Nucleic Acid

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A “nucleic acid” may comprise any part of a gene sequence, of from about 2 nucleotides to the full length of a peptide or polypeptide encoding region. For example, a nucleic acid may comprise part of a therapeutic gene sequence.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In certain embodiments, the nucleic acid is an RNA molecule. For example, the RNA molecule can be a messenger RNA (mRNA) molecule. In other embodiments, the RNA molecule is an interfering RNA. RNA interference (RNA₁) is a form of gene silencing triggered by double-stranded RNA (dsRNA). DsRNA activates post-transcriptional gene expression surveillance mechanisms that appear to function to defend cells from virus infection and transposon activity. Fire et al. (1998); Grishok et al. (2000); Ketting et al. (1999); Lin & Avery (1999); Montgomery et al. (1998); Sharp (1999); Sharp & Zamore (2000); Tabara et al. (1999). Activation of these mechanisms targets mature, dsRNA-complementary mRNA for destruction. RNAᵢ offers major experimental advantages for study of gene function. These advantages include a very high specificity, ease of movement across cell membranes, and prolonged down-regulation of the targeted gene. Fire et al. (1998); Grishok et al. (2000); Ketting et al. (1999); Lin & Avery (1999); Montgomery et al. (1998); Sharp (1999); Sharp & Zamore (2000); Tabara et al. (1999). RNAᵢ also is incredibly potent. It has been estimated that only a few copies of dsRNA are required to knock down >95% of targeted gene expression in a cell. Fire et al. (1998). Moreover, dsRNA has been shown to silence genes in a wide range of systems, including plants, protozoans, C. elegans and Drosophila. Grishok et al. (2000); Sharp (1999); Sharp & Zamore (1999).

2. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Table 1 below lists non-limiting, purine and pyrimidine derivatives and analogs.

TABLE 1
Purine and Pyrmidine Derivatives or Analogs

| Abbr. | Modified base description | Abbr. | Modified base description |
|---|---|---|---|
| ac4c | 4-acetylcytidine | Mam5s2u | 5-methoxyaminomethyl-2-thiouridine |
| Chm5u | 5-(carboxyhydroxylmethyl) uridine | Man q | Beta,D-mannosylqueosine |
| Cm | 2′-O-methylcytidine | Mcm5s2u | 5-methoxycarbonylmethyl-2-thiouridine |
| Cmnm5s2u | 5-carboxymethylamino-methyl-2-thioridine | Mcm5u | 5-methoxycarbonylmethyluridine |
| Cmnm5u | 5-carboxymethylaminomethyluridine | Mo5u | 5-methoxyuridine |
| D | Dihydrouridine | Ms2i6a | 2-methylthio-N6-isopentenyladenosine |
| Fm | 2′-O-methylpseudouridine | Ms2t6a | N-((9-beta-D-ribofuranosyl-2-methylthiopurine-6- |
| | | | yl)carbamoyl)threonine |
| Gal q | Beta,D-galactosylqueosine | Mt6a | N-((9-beta-D-ribofuranosylpurine-6-yl)N-methyl- |
| | | | carbamoyl)threonine |
| Gm | 2′-O-methylguanosine | Mv | Uridine-5-oxyacetic acid methylester |
| I | Inosine | o5u | Uridine-5-oxyacetic acid (v) |
| I6a | N6-isopentenyladenosine | Osyw | Wybutoxosine |
| m1a | 1-methyladenosine | P | Pseudouridine |
| m1f | 1-methylpseudouridine | Q | Queosine |
| m1g | 1-methylguanosine | s2c | 2-thiocytidine |
| m1I | 1-methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | T | 5-methyluridine |
| m3c | 3-methylcytidine | t6a | N-((9-beta-D-ribofuranosylpurine-6- |
| | | | yl)carbamoyl)threonine |
| m5c | 5-methylcytidine | Tm | 2′-O-methyl-5-methyluridine |
| m6a | N6-methyladenosine | Um | 2′-O-methyluridine |
| m7g | 7-methylguanosine | Yw | Wybutosine |
| Mam5u | 5-methylaminomethyluridine | X | 3-(3-amino-3-carboxypropyl)uridine, (acp3)u |

A nucleobase may be comprised in a nucleoside or nucleotide, using any chemical or natural synthesis method described herein or known to one of ordinary skill in the art.

3. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

4. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

5. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as flourescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonuceotides to enhanced their membrane permeability and stability; U.S. Pat. No. 5,214,136 which describes olignucleotides conjugaged to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

6. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

7. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

8. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

9. Antisense Nucleic Acids

As used herein, the terms “antisense” or “complementary” mean nucleic acids that are substantially complementary over their entire length and have very few base mismatches. The nucleic acids may be DNA or RNA molecules.

A nucleic acid “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

For example, sequences of fifteen bases in length may be termed complementary when they have a complementary nucleotide at thirteen or fourteen positions out of fifteen. Naturally, sequences which are “completely complementary” will be sequences which are entirely complementary throughout their entire length and have no base mismatches.

In certain embodiments, a “complementary” nucleic acid comprises a sequence in which about 70% to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

Other sequences with lower degrees of homology also are contemplated. For example, an antisense construct which has limited regions of high homology, but also contains a non-homologous region (e.g., a ribozyme) could be designed. These molecules, though having less than 50% homology, would bind to target sequences under appropriate conditions.

The polynucleotides according to the present invention may encode a particular gene or portion of a gene that is sufficient to effect antisense inhibition of protein expression. The polynucleotides may be derived from genomic DNA, i.e., cloned directly from the genome of a particular organism. In other embodiments, however, the polynucleotides may be complementary DNA (cDNA). cDNA is DNA prepared using messenger RNA (mRNA) as template. Thus, a cDNA does not contain any interrupted coding sequences and usually contains almost exclusively the coding region(s) for the corresponding protein. In other embodiments, the antisense polynucleotide may be produced synthetically.

It may be advantageous to combine portions of the genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

As stated above, although the antisense sequences may be full length genomic or cDNA copies, or large fragments thereof, they also may be shorter fragments, or “oligonucleotides,” defined herein as polynucleotides of 50 or less bases. Although shorter oligomers (8-20) are easier to make and increase in vivo accessibility, numerous other factors are involved in determining the specificity of base-pairing. For example, both binding affinity and sequence specificity of an oligonucleotide to its complementary target increase with increasing length. It is contemplated that oligonucleotides of 8, 9, 10, 11, 12, 13, 14, 15, 16, 17, 18, 19, 20, 25, 30, 35, 40, 45 or 50 base pairs will be used. While all or part of the gene sequence may be employed in the context of antisense construction, statistically, any sequence of 17 bases long should occur only once in the human genome and, therefore, suffice to specify a unique target sequence.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

As an alternative to targeted antisense delivery, targeted ribozymes may be used. The term “ribozyme” refers to an RNA-based enzyme capable of targeting and cleaving particular base sequences in both DNA and RNA. Ribozymes can either be targeted directly to cells, in the form of RNA oligonucleotides incorporating ribozyme sequences, or introduced into the cell as an expression vector encoding the desired ribozymal RNA. Ribozymes may be used and applied in much the same way as described for antisense polynucleotide. Ribozyme sequences also may be modified in much the same way as described for antisense polynucleotide. For example, one could incorporate non-Watson-Crick bases, or make mixed RNA/DNA oligonucleotides, or modify the phosphodiester backbone.

Alternatively, the antisense oligo- and polynucleotides according to the present invention may be provided as mRNA via transcription from expression constructs that carry nucleic acids encoding the oligo- or polynucleotides. Throughout this application, the term “expression construct” is meant to include any type of genetic construct containing a nucleic acid encoding an antisense product in which part or all of the nucleic acid sequence is capable of being transcribed. Typical expression vectors include bacterial plasmids or phage, such as any of the pUC or Bluescript™ plasmid series or, as discussed further below, viral vectors adapted for use in eukaryotic cells.

10. Therapeutic Gene

The term “gene” is used for simplicity to refer to a functional protein, polypeptide, or peptide-encoding unit. “Therapeutic gene” is a gene which can be administered to a subject for the purpose of treating or preventing a disease. For example, a therapeutic gene can be a gene administered to a subject for treatment or prevention of cancer. Examples of classes of therapeutic genes include tumor suppressor genes, genes that induce apoptosis, genes encoding enzymes, genes encoding antibodies, and genes encoding hormones. Examples of therapeutic genes include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, FUS1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include the tumor suppressor genes at 3p21.3, including FUS1, Gene 26 (CACNA2D2), PL6, Beta*(BLU), LUCA-1 (HYAL1), LUCA-2 (HYAL2), 123F2 (RASSF1), 101F6, Gene 21 (NPRL2), and SEM A3. These genes, which play a major role in the pathogenesis of human lung cancer and other cancers, are addressed in detail in U.S. Patent Application. Pub. No. 20040016006 and U.S. Patent Application Pub. No. 20020164715, each of which is herein specifically incorporated by reference in its entirety.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase.

Therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, α-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

As will be understood by those in the art, the term “therapeutic gene” includes genomic sequences, cDNA sequences, and smaller engineered gene segments that express, or may be adapted to express, proteins, polypeptides, domains, peptides, fusion proteins, and mutants that maintain some or all of the therapeutic function of the full length protein encoded by the therapeutic gene. For example, a chromosome 3p21.3 tumor suppressor gene, such as a “FUS1” therapeutic gene, includes genomic sequences, cDNA sequences, and smaller engineered gene segments that express, or may be adapted to express, a protein, polypeptide, domain, peptide, fusion protein or mutant FUS1 that maintains some or all of the function of full-length FUS1 protein. As set forth above, these genes are discussed in greater detail in U.S. Patent Application Pub. No. 20040016006 and U.S. Patent Application Pub. No. 20020164715, each of which is herein specifically incorporated by reference in its entirety.

Therapeutic genes also include antisense nucleic acids and interfering RNA, both of which are discussed in other parts of this specification. The nucleic acid molecule encoding a therapeutic gene may comprise a contiguous nucleic acid sequence that is about 5 to 12,000 or more nucleotides, nucleosides, or base pairs in length.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

Encompassed within the definition of “therapeutic gene” is a “biologically functional equivalent” therapeutic gene. Accordingly, sequences that have about 70% sequence homology to about 99% sequence homology and any range or amount of sequence homology derivable therein, such as, for example, about 70% to about 80%, and more preferably about 81% and about 90%; or even more preferably, between about 91% and about 99%; of amino acids that are identical or functionally equivalent to the amino acids of the therapeutic gene will be sequences that are biologically functional equivalents provided the biological activity of the protein is maintained.

In particular embodiments, the invention concerns isolated nucleic acid segments and recombinant vectors incorporating DNA sequences that encode one or more therapeutic genes. Vectors of the present invention are designed, primarily, to transform cells with a therapeutic gene under the control of regulated eukaryotic promoters (i.e., inducible, repressable, tissue specific). Also, the vectors may contain a selectable marker if, for no other reason, to facilitate their manipulation in vitro. However, selectable markers may play an important role in producing recombinant cells.

Tables 2 and 3, below, list a variety of regulatory signals for use according to the present invention.

TABLE 2
Inducible Elements

| Element | Inducer | References |
|---|---|---|
| MT II | Phorbol Ester (TPA) | Palmiter et al., 1982; |
| | Heavy metals | Haslinger and Karin, 1985; |
| | | Searle et al., 1985; Stuart et |
| | | al., 1985; Imagawa et al., |
| | | 1987; Karin et al., 1987; |
| | | Angel et al., 1987b; |
| | | McNeall et al., 1989 |
| MMTV (mouse | Glucocorticoids | Huang et al., 1981; Lee et al., |
| mammary tumor | | 1981; Majors and Varmus, |
| virus) | | 1983; Yamamoto et al., 1983; |
| | | Lee et al., 1984; Ponta et al., |
| | | 1985; Si.e., i et al., 1986 |
| β-Interferon | poly(rI)X | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TFA) | Angel et al., 1987b |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb HSP70 | Ela, SV40 Large T | Taylor et al., 1989; Taylor |
| | Antigen | and Kingston, 1990a, b |
| Proliferin | Phorbol Ester-TPA | Mordacq and Linzer, 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

TABLE 3
Other Promoter/Enhancer Elements

| Promoter/Enhancer | References |
|---|---|
| Immunoglobulin Heavy Chain | Banerji et al., 1983; Gillies et al., 1983; Grosschedl |
| | and Baltimore, 1985; Atchinson and Perry, 1986, |
| | 1987; Imler et al., 1987; Neuberger et al., 1988; |
| | Kiledjian et al., 1988; |
| Immunoglobulin Light Chain | Queen and Baltimore, 1983; Picard and Schaffner, |
| | 1985 |
| T-Cell Receptor | Luria et al., 1987, Winoto and Baltimore, 1989; |
| | Redondo et al., 1990 |
| HLA DQ α and DQ β | Sullivan and Peterlin, 1987 |
| β-Interferon | Goodbourn et al., 1986; Fujita et al., 1987; |
| | Goodbourn and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRα | Sherman et al., 1989 |
| β-Actin | Kawamoto et al., 1988; Ng et al., 1989 |
| Muscle Creatine Kinase | Jaynes et al., 1988; Horlick and Benfield, 1989; |
| | Johnson et al., 1989a |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987, Tronche et al., 1989, 1990 |
| α-Fetoprotein | Godbout et al., 1988; Campere and Tilghman, |
| | 1989 |
| γ-Globin | Bodine and Ley, 1987; Perez-Stable and |
| | Constantini, 1990 |
| β-Globin | Trudel and Constantini, 1987 |
| c-fos | Cohen et al., 1987 |
| c-HA-ras | Triesman, 1985; Deschamps et al., 1985 |
| Insulin | Edlund et al., 1985 |
| Neural Cell Adhesion Molecule | Hirsch et al., 1990 |
| (NCAM) | |
| a₁₋Antitrypain | Latimer et al., 1990 |
| H2B (TH2B) Histone | Hwang et al., 1990 |
| Mouse or Type I Collagen | Rippe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh |
| | and Lockett, 1985; Firak and Subramanian, 1986; |
| | Herr and Clarke, 1986; Imbra and Karin, 1986; |
| | Kadesch and Berg, 1986; Wang and Calame, 1986; |
| | Ondek et al., 1987; Kuhl et al., 1987 Schaffner et |
| | al., 1988 |
| Polyoma | Swartzendruber and Lehman, 1975; Vasseur et al., |
| | 1980; Katinka et al., 1980, 1981; Tyndell et al., |
| | 1981; Dandolo et al., 1983; Hen et al., 1986; Si.e., i |
| | et al., 1988; Campbell and Villarreal, 1988 |
| Retroviruses | Kriegler and Botchan, 1983; Kriegler et al., |
| | 1984a, b; Bosze et al., 1986; Miksicek et al., 1986; |
| | Celander and Haseltine, 1987; Thiesen et al., 1988; |
| | Celander et al., 1988; Chol et al., 1996; Reisman |
| | and Rotter, 1989 |
| Papilloma Virus | Campo et al., 1983; Lusky et al., 1983; Spandidos |
| | and Wilkie, 1983; Spalholz et al., 1985; Lusky and |
| | Botchan, 1986; Cripe et al., 1987; Gloss et al., |
| | 1987; Hirochika et al., 1987, Stephens and |
| | Hentschel, 1987 |
| Hepatitis B Virus | Bulla and Siddiqui, 1988; Jameel and Siddiqui, |
| | 1986; Shaul and Ben-Levy, 1987; Spandau and |
| | Lee, 1988 |
| Human Immunodeficiency Virus | Muesing et al., 1987; Hauber and Cullan, 1988; |
| | Jakobovits et al., 1988; Feng and Holland, 1988; |
| | Takebe et al., 1988; Berkhout et al., 1989; Laspia |
| | et al., 1989; Sharp and Marciniak, 1989; Braddock |
| | et al., 1989 |
| Cytomegalovirus | Weber et al., 1984; Boshart et al., 1985; Foecking |
| | and Hofstetter, 1986 |
| Gibbon Ape Leukemia Virus | Holbrook et al., 1987; Quinn et al., 1989 |

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term “promoter” will be used here to refer to a group of transcriptional control modules that are clustered around the initiation site for RNA polymerase II.

Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation.

Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way. One of ordinary skill in the art would be familiar with promoters and enhancers, and their applications.

Another signal that may prove useful is a polyadenylation signal. Such signals may be obtained from the human growth hormone (hGH) gene, the bovine growth hormone (BGH) gene, or SV40.

The use of internal ribosome binding sites (IRES) elements are used to create multigene, or polycistronic, messages. IRES elements are able to bypass the ribosome scanning model of 5-methylatd cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In any event, it will be understood that promoters are DNA elements which when positioned functionally upstream of a gene leads to the expression of that gene. Most transgene constructs of the present invention are functionally positioned downstream of a promoter element.

B. Lipid Compositions

In certain embodiments, the present invention concerns methods to prevent or reduce inflammation secondary to administration of a lipid-nucleic acid complex in a subject. The invention also concerns methods of screening for inhibitors of the inflammatory response associated with administration of a lipid-nucleic acid complex to a subject. In addition, the present invention concerns compositions that include a lipid, a nucleic acid, and non-steroidal anti-inflammatory agent, a salicylate, an anti-rheumatic agent, an antihistamine, or an immunosuppressive agent. Each of these aspects of the present invention pertains to lipids and/or lipid compositions.

A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

1. Lipid Types

A neutral fat may comprise a glycerol and a fatty acid. A typical glycerol is a three carbon alcohol. A fatty acid generally is a molecule comprising a carbon chain with an acidic moiety (e.g., carboxylic acid) at an end of the chain. The carbon chain may of a fatty acid may be of any length, however, it is preferred that the length of the carbon chain be of from about 2 to about 30 or more carbon atoms, and any number or range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

2. Charged and Neutral Lipid Compositions

In certain embodiments, a lipid component of a composition is uncharged or primarily uncharged. In one embodiment, a lipid component of a composition comprises one or more neutral lipids. For example, the neutral lipid may be DOPE. In other embodiments of the present invention, the lipid is a cationic lipid. Examples of cationic lipids are discussed elsewhere in this specification. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

3. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

4. Lipid Composition Structures

In certain embodiments of the present invention, the lipid is associated with a nucleic acid. A nucleic acid associated with a lipid may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. A lipid or lipid/nucleic acid-associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure.

In certain embodiments, a lipid composition may comprise anywhere from about 1% to about 100%, or any percent derivable therein, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a drug. Thus, it is contemplated that lipid compositions of the present invention may comprise any of the lipids, lipid types or other components in any combination or percentage range.

a. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modern Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

b. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; E1-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

5. Liposomes

In particular embodiments, a lipid comprises a liposome. Liposomes are discussed in greater detail in the Summary of the Invention and in other parts of this specification.

In certain embodiments, phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition or a liposome, because of the instability and leakiness of the resulting liposomes.

In particular embodiments, a lipid and/or nucleic acid may be, for example, encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, etc.

a. Making Liposomes

A liposome used according to the present invention can be made by different methods, as would be known to one of ordinary skill in the art. Phospholipids can form a variety of structures other than liposomes when dispersed in water, depending on the molar ratio of lipid to water. At low ratios the liposome is the preferred structure.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the nucleic acid, and/or other component(s). Additional components may or may not include a non-steroidal anti-inflammatory agent, a salicylate, an anti-rheumatic agent, an antihistamine, and/or an immunosuppressive agent. Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the lipid-nucleic acid complexes of the present invention may be about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios. One of ordinary skill in the art would be familiar with the wide range of techniques available for preparing liposomes.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomes of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs. Liposomal encapsulation has resulted in a lower toxicity and a longer serum half-life for such compounds (Gabizon et al., 1990).

Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Mayer et al., 1985; Mayhew et al. 1987; Mayhew et al., 1984; Cheng et al., 1987; and Liposome Technology, 1984, each incorporated herein by reference).

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

b. Liposome Targeting

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/nucleic acid composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of nucleic acid. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

i. Cross-linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 4 details certain hetero-bifunctional cross-linkers considered useful in the present invention.

TABLE 4
Hetero-Bifunctional Cross-Linkers

| | | | Spacer |
|---|---|---|---|
| | | | Arm |
| | | | Length\ |
| | | | after |
| | Reactive | | cross- |
| Linker | Toward | Advantages and Applications | linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC- | Primary amines | Extended spacer arm | 15.6 A |
| SPDP | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein | |
| | | conjugation | |
| Sulfo- | Primary amines | Stable maleimide reactive group | 11.6 A |
| SMCC | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein | |
| | | conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo- | Primary amines | Extended spacer arm | 14.5 A |
| SMPB | Sulfhydryls | Water-soluble | |
| EDC/Sulfo- | Primary amines | Hapten-Carrier conjugation | 0 |
| NHS | Carboxyl | | |
| | groups | | |
| ABH | Carbohydrates | Reacts with sugar groups | 11.9 A |
| | Nonselective | | |

In instances where a particular polypeptide does not contain a residue amenable for a given cross-linking reagent in its native sequence, conservative genetic or synthetic amino acid changes in the primary sequence can be utilized.

ii. Targeting Ligands

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see Heath et al., 1986) For example, disialoganglioside GD2 is a tumor antigen that has been identified neuroectodermal origin tumors, such as neuroblastoma, melanoma, small-cell lung carcenoma, glioma and certain sarcomas (Mujoo et al., 1986, Schulz et al., 1984). Liposomes containing anti-disialoganglioside GD2 monoclonal antibodies have been used to aid the targeting of the liposomes to cells expressing the tumor antigen (Montaldo et al., 1999; Pagnan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, a lipid-nucleic acid complex may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific delivery and/or targeting vehicle may comprise a specific binding ligand in combination with a liposome. The nucleic acid to be delivered is housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

c. Nanoparticles

In certain embodiments, the lipids of the present invention are comprised in a nanoparticle. A nanoparticle is herein defined as a submicron particle. For example, the nanoparticle may have a diameter of from about 1 to about 100 nanometers. The particle can be composed of any material or compound. In the context of the present invention, for example, a “nanoparticle” may include certain liposomes that have a diameter of from about 1 to about 100 nanometers.

d. Liposome/Nucleic Acid Combinations

In certain embodiments, a liposome may include a nucleic acid, such as, for example, an oligonucleotide, a polynucleotide or a nucleic acid construct (e.g., an expression vector). Where a bacterial promoter is employed in the DNA construct that is to be transfected into eukaryotic cells, it also will be desirable to include within the liposome an appropriate bacterial polymerase.

It is contemplated that when the liposome composition comprises a cell or tissue specific nucleic acid, this technique may have applicability in the present invention. In certain embodiments, lipid-based non-viral formulations provide an alternative to viral gene therapies. An exemplary method for targeting viral particles to cells that lack a single cell-specific marker has been described (U.S. Pat. No. 5,849,718). In this method, for example, antibody A may have specificity for tumor, but also for normal heart and lung tissue, while antibody B has specificity for tumor but also normal liver cells. The use of antibody A or antibody B alone to deliver an anti-proliferative nucleic acid to the tumor would possibly result in unwanted damage to heart and lung or liver cells. However, antibody A and antibody B can be used together for improved cell targeting. Thus, antibody A is coupled to a gene encoding an anti-proliferative nucleic acid and is delivered, via a receptor mediated uptake system, to tumor as well as heart and lung tissue. However, the gene is not transcribed in these cells as they lack a necessary transcription factor. Antibody B is coupled to a universally active gene encoding the transcription factor necessary for the transcription of the anti-proliferative nucleic acid and is delivered to tumor and liver cells. Therefore, in heart and lung cells only the inactive anti-proliferative nucleic acid is delivered, where it is not transcribed, leading to no adverse effects. In liver cells, the gene encoding the transcription factor is delivered and transcribed, but has no effect because no an anti-proliferative nucleic acid gene is present. In tumor cells, however, both genes are delivered and the transcription factor can activate transcription of the anti-proliferative nucleic acid, leading to tumor-specific toxic effects.

The addition of targeting ligands for gene delivery for the treatment of hyperproliferative diseases permits the delivery of genes whose gene products are more toxic than do non-targeted systems. Examples of therapeutic genes are discussed in other sections of this specification.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene such as a tumor suppressor gene or anti-oncogene.

C. Selected Anti-Inflammatory Agents

The present invention pertains to methods and compositions that involve either one or more members of the group consisting of a non-steroidal anti-inflammatory agent, a salicylate, an anti-rheumatic agent, an antihistamine, or an immunosuppressive agent. These agents have in common the fact that they can function to decrease the signs and symptoms of inflammation when administered to a subject. A wide variety of anti-inflammatory agents are known to those of ordinary skill in the art. Some of the major classes of anti-inflammatory agents include the following classes of agents.

1. Non-Steroidal Anti-Inflammatory Agents

Non-steroidal anti-inflammatory agents include a class of drugs used in the treatment of inflammation and pain. The exact mode of action of this class of drugs is unknown. Examples of members of this class of agents include, but are not limited to, ibuprofen, ketoprofen, nabumetone, piroxicam, naproxen, diclofenac, indomethacin, sulindac, tolmetin, etodolac, oxaprozin, rofecoxib, and celecoxib.

Theories of the mechanism of action of these agents include (1) inhibition of cyclooxygenase activity and prostaglandin activity, (2) inhibition of lipoxygenase, leukotriene synthesis, lysosomal enzyme release, and neutrophil aggregation, and (3) decrease in Rh factor. Also included in the definition of non-steroidal anti-inflammatory agents are the proprionic acid derivatives, acetic acids, oxicam derivatives, fenamates, and the COX II inhibitors.

The proprionic acid derivatives include ibuprofen, fenoprofen, fluorbiprofen, ketoprofen, naproxen, naproxen sodium, and oxaprozin. These agents are reversible inhibitors of cyclooxygenase.

The acetic acids function by reversibly inhibiting cyclooxygenase. Acetic acids include diclofenac, diclofenac sodium and misoprostol, ketorolac, tolmetin, indomethacin, sulindac, and etodolac.

The mechanism of action of the oxicam derivatives is unknown. Oxicam derivatives are used in the acute and long-term treatment of rheumatoid arthritis. Piroxicam is one example of an oxicam derivative. The fenamates have no advantage over other non-steroidal anti-inflammatory agents.

Fenamates are prescribed for the treatment of dysmenorrhea. Examples of fenamates include mefanamic acid and mclofenamate.

The COX II inhibitors are selective to COX II, therefore they have no adverse effects on the GI system or the kidneys. The relieve pain in the same manner as other non-steroidal anti-inflammatory agents. Celecoxib and Vioxx are examples of COX II inhibitors.

One of ordinary skill in the art would be familiar with the numerous agents that are members of this class, as well as the properties and indications for the use of this class of agents.

2. Salicylates

Included in this category are salicylates and derivates of salicylates. Aspirin (acetylsalicylic acid) and other salicylates work directly be irreversibly inactivating cyclooxygenase. The drugs act to depress painful stimuli at the thalamus and hypothalamus. Sodium salicylate, choline salicylate, and choline magnesium salicylate are reversible inhibitors of cyclooxygenase. They prevent sensitization of pain receptors to both mechanical and chemical stimuli. One of ordinary skill in the art would be familiar with this class of agents.

Diflunisal is an example of a derivative of salicylic acid which does not metabolize to salicylate and therefore cannot cause salicylate intoxication. It is a peripherally-active non-narcotic analgesic with anti-inflammatory properties. Diflunisal is three to four times more potent than aspirin as an analgesic and an anti-inflammatory, but it has no antipyretic properties because it does not enter the central nervous system. It has fewer side effects than other salicylates.

Salicylates are indicated for relief of pain, rheumatoid arthritis, and osteoarthritis. Potential adverse reactions include tinnitus and central hyperventilation.

3. Anti-Rheumatic Agents

The anti-rheumatic agents include a diverse group of agents that are primarily used in the treatment of rheumatoid arthritis, and that are not non-steroidal anti-inflammatory agents, salicylates, immunosuppressive agents, or steroids. One of ordinary skill in the art would be familiar with this group of agents, and the indications for use of these agents.

Included in this class of agents are the slow-acting anti-rheumatic agents, such as gold salts. Examples of gold salts include gold sodium thiomalate, aurothioglucose, and auranofin. Gold salts are taken up by macrophages, with suppression of phagocytosis and lysosomal enzyme activity. This in turn slows down the process of bone and articular destruction. Gold salts are indicated in the treatment of progressing rheumatoid arthritis when prolonged therapy is required.

The anti-rheumatic agents also include chloroquine and hydroxychloroquine. These agents are anti-malarial agents which are indicated in the treatment of rheumatoid arthritis in its severe stages. The actual mechanism of these drugs is unknown. The treatment effects of these drugs may not be seen for up to six months. Adverse effects are common and severe, and include GI disturbances, skin rashes, muscular weakness, and irreversible retinal damage and headaches.

Penicillamine is another example of an anti-rheumatic agent. It is an analogue of the amino acid cysteine. The mode of action of penicillamine is unknown. However, Rh factor levels have been proven to fall with its administration. Penicillamine slows the progression of bone destruction and rheumatoid arthritis. Response to therapy may take up to 2 to 3 months. Adverse effects with prolonged treatment include severe skin reactions, nephritis, and aplastic anemia. Penicillamine is used after gold salts have failed and before corticosteroids have been attempted.

Other members of this group include leflunomide, a pyrimidine analogue which incorporates itself into the DNA of T lymphocytes and inhibits the inflammatory response. Etanercept and infliximab are two other anti-rheumatic agents which inhibit the action of tumor necrosis factor. Other members of this class include CD4 monoclonoal antibody agents. These agents have a similar action to leflunomide, but are specific to the CD4 cells responsible for the immune response in rheumatoid arthritis and therefore lack the side effects seen with other chemotherapeutic agents which nonselectively affect all rapidly reproducing cells.

4. Antihistamines

Antihistamines are reversible competitive antagonists of histamine at H₁ receptor sites. They do not prevent histamine release or bind to the histamine that has already been released. The H₁ receptor blockade results in decreased vascular permeability, reduction of pruritus and relaxation of smooth muscle in the respiratory and gastrointestinal tracts. Antihistamines are clinically useful in alleviating symptoms that are attributed to the early-phase allergic reaction, such as rhinorrhea, pruritus, and sneezing. One of ordinary skill in the art would be familiar with this class of agents, as well as the mechanism of action of these agents, and the indications for use of these agents.

Although very effective, the first-generation antihistamines such as diphenhydramine, chlorpheniramine, clemastine, hydroxyzine, and triprolidine may cause sedation and anticholinergic side-effects. The second generation antihistamines, including astemizole, terfenadine, loratadine, cetirizine, and fexofenadine, have been known to minimize these side effects. Terfenadine and astemizole were removed from the market due to serious cardiovascular side effects. The newest antihistamine agent, desloratadine, an active metabolite of loratadine, has been categorized as a third-generation antihistamine.

First-generation antihistamines are used in the treatment of hypersensitivity reactions, type 1, including perennial or seasonal allergic rhinitis, vasomotor rhinitis, allergic conjunctivitis, and urticaria. Diphenhydramine is also commonly used as an anti-tussive, sleep aid, anti-Parkinsonism, and for motion sickness. Hydroxyzine has been used as a sedative and as an anti-emetic agent. Promethazine is used for motion sickness, sedation, or analgesia.

5. Immunosuppressive Agents

These agents include a class of drugs that, in general, are used in the treatment of inflammatory conditions such as rheumatoid arthritis when treatment with non-steroidal anti-inflammatory agents and other anti-rheumatic agents have failed. Immunosuppressive agents have a stabilizing effect on the immune system. Examples of agents in this class include methotrexate, mechlorethamine, cyclophosphamide, chlorambucil, cyclosporine, and azathioprine. One of ordinary skill in the art would be familiar with these agents, and other members of this class of agents, as well as the mechanism of actions of these agents and indications for use of these agents.

Methotrexate is an anti-folate purine analogue that is often used in chemotherapy. The exact mode of action is unknown, but it is believed to act as a purine analogue by incorporating itself into the DNA structure of the inflammatory cells to disrupt their ability to reproduce. Methotrexate is indicated for severe, active rheumatoid arthritis only when non-steroidal anti-inflammatory agents and other anti-rheumatic agents have failed. Effects are seen 3 to 6 weeks after administration. Side effects include hepatotoxicity, fibrosis, cirrhosis, anemia, leukopenia, thrombocytopenia, and GI problems.

Cyclosporine is another member of this class of agents. In the cytoplasm of a cell, cyclosporine binds to its immunophilin, cyclophylin, forming a complex between cyclosporin and the cyclophylin. The cyclosporine-cyclophylin complex binds and blocks the function of the enzyme calcineurin, which as a serine-threonine phosphatase activity. As a result, calcineurin fails to dephosphorylate the cytoplasmic component of the nuclear factor of activated T cells. Calcineurin also fails to transport the nuclear factor of activated T cells to the nucleus, and nuclear factor of activated T cells fails to bind the nuclear component of the nuclear factor of activated T cells. As a result, IL-2 production is initiated. Consequently, T cells do not produce IL-2, which is necessary for full T-cell activation. As a result, T cell activation is inhibited.

D. Screening Methods

The present invention includes embodiments that provide for methods of screening for inhibitors of the inflammatory response associated with administration of a lipid-nucleic acid complex to a subject. Any type of subject is contemplated by the present invention. For example, in some embodiments, the subject is a human subject.

Any assay techniques known to those of skill in the art are contemplated by the present invention. For example, the assays may comprise random high-throughput screening of large libraries of candidate substances. Alternatively, the assays may be used to focus on particular classes of compounds selected with an eye towards structural attributes that are believed to make them more likely to prevent or inhibit the inflammation associated with administration of a lipid-nucleic acid complex. The assays involved in these screening methods may include cell-free assays, in vitro assays, in cyto assays, in vivo assays, or any assay technique known to those of skill in the art.

An inhibitor of the inflammatory response associated with administration of a lipid-nucleic acid complex is any substance that can diminish the inflammatory response associated with administration of a lipid-nucleic acid complex to a subject. The method of screening for inhibitors of the inflammatory response associated with administration of a lipid-nucleic acid complex to a subject generally comprises:
(a) providing a candidate substance suspected of preventing or inhibiting the inflammation association with administration of a lipid-nucleic acid complex;
(b) contacting a composition comprising the lipid-nucleic acid complex and the candidate substance with the subject; and
(c) assesssing inflammation in the subject.
   The candidate substance can be a candidate substance suspected of either inhibiting or preventing the inflammatory response associated with administration of a lipid-nucleic acid complex.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially prevent or inhibit the inflammation associated with administration of a lipid-nucleic acid complex. The candidate substance may be a protein or fragment thereof, a small molecule, an antibody, or even a polynucleotide. It may prove to be the case that the most useful pharmacological compounds will be compounds that are structurally related to known anti-inflammatory agents, such as non-steroidal anti-inflammatory agents, salicylates, anti-rheumatic agents, antihistamines, and immunosuppressive agents. Using lead compounds to help develop improved compounds is known as “rational drug design” and includes not only comparisons with known inhibitors and activators, but predictions relating to the structure of target molecules.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single-chain antibodies or expression constructs coding thereof), each of which would be specific for a given target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An agent that prevents or inhibits the inflammation associated with administration of a lipid-nucleic acid complex may, according to the present invention, be one which exerts its effect upstream, downstream or directly on a known pathway involved in the inflammatory response. Regardless of the type of agent identified by the present screening methods, the effect of the inhibition or prevention by such a compound results in an inhibition of the inflammation associated with administration of a lipid-nucleic acid complex.

2. In Vitro Assays

A quick, inexpensive and easy assay to run is an in vitro assay. Such assays can be run quickly and in large numbers, thereby increasing the amount of information obtainable in a short period of time. A variety of vessels may be used to run the assays, including test tubes, plates, dishes and other surfaces such as dipsticks or beads. One example of a cell free assay is a binding assay. While not directly addressing inhibition of an inflammatory response in a subject, the ability of a candidate substance to inhibit the inflammatory response in vitro may be strong evidence of a related biological effect on the subject. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564, U.S. Pat. No. 6,457,809, U.S. Pat. No. 6,406,921, and U.S. Pat. No. 5,994,131. Large numbers of small peptide test compounds are synthesized on a solid substrate, such as plastic or some other surface. Bound polypeptide is detected by various methods.

3. In Cyto Assays

Various cell lines can be utilized for screening assays, including cells specifically engineered for this purpose. Examples of cells used in the screening assays include cancer cells, cells infected with a virus, foam cells, macrophages, neuronal cells or dendritic cells. The cell may be a stimulated cell, such as a cell stimulated with a growth factor. One of skill in the art would understand that the invention disclosed herein contemplates a wide variety of in cyto assays for measuring parameters that correlate with inhibition of the inflammatory response associated with administration of a lipid-nucleic acid complex.

Depending on the assay, culture may be required. The cell may be examined using any of a number of different physiologic assays to assess for inhibition of inflammation. Alternatively, molecular analysis may be performed, for example, looking at protein expression, mRNA expression (including differential display of whole cell or polyA RNA) and other parameters associated with inflammatory pathways.

4. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies an inhibitor of the inflammatory response associated with administration of a lipid-nucleic acid complex.

Treatment of animals with test compounds will involve the administration of the compound, in an appropriate form, to the animal. Any animal model of cancer known to those of skill in the art can be used in the screening techniques of the present invention. Administration will be by any route that could be utilized for clinical or non-clinical purposes, including but not limited to oral, nasal, buccal, intratumoral, or even topical. Alternatively, administration may be by intratracheal instillation, bronchial instillation, intradermal, subcutaneous, intramuscular, intraperitoneal. inhalation or intravenous injection.

Determining the effectiveness of a compound in vivo may involve a variety of different criteria. Also, measuring toxicity and dose response can be performed in animals in a more meaningful fashion than in in vitro or in cyto assays.

In some embodiments, the subject is a human subject.

Any method of assaying for inflammation is contemplated by the present invention. One of ordinary skill in the art would be familiar with the wide range of techniques available for assaying for inflammation in a subject, whether that subject is an animal or a human subject. For example, the assay may involve measurement of particular biochemical factors associated with inflammation, such as C-reactive protein or NF-κB levels. The assays may involve the measurement of inflammatory mediators in particular cell types, such as T cells, B cells, neutrophils, and macrophages. Alternatively, assaying for inflammation may include assays based on clinical response, such as measurement of the size of an area of erythema at a particular site following administration of a lipid-nucleic acid complex or measurement of a particular inflammatory cell response following administration of a lipid-nucleic acid complex. One of ordinary skill in the art would be familiar with the wide range of assays available for measurement of an inflammatory response in a subject. For example, assays may involve measurement of cytokine levels, prostaglandin levels, or COX-2 levels in body fluids such as blood, saliva, urine, bronchial lavage fluid, ascites fluid, etc.

E. Pharmaceutical Preparations and Therapeutic Methods

1. Overview

The present invention concerns compositions that include (1) a lipid, (2) nucleic acid, and (3) a non-steroidal anti-inflammatory agent, a salicylate, an anti-rheumatic agent, an antihistamine, or an immunosuppreessive agent.

2. Pharmaceutical Preparations

In certain embodiments of the present invention, the compositions are aqueous compositions. Aqueous compositions of the present invention comprise an effective amount of a lipid, a nucleic acid, and a non-steroidal anti-inflammatory agent, a salicylate, an anti-rheumatic agent, an antihistamine, or an immunosuppreessive agent, dissolved or dispersed in a pharmaceutically acceptable carrier or aqueous medium. The phrases “pharmaceutical preparation” or “pharmacologically effective” or “pharmaceutically acceptable” refer to molecular entities and compositions that do not produce an adverse, allergic or other untoward reaction when administered to an animal, or a human, as appropriate.

Certain embodiments of the present compositions include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition containing an active agent of the invention disclosed herein as a component or active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An agent or substance of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The active agents disclosed herein may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include vaginal suppositories and pessaries.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

3. Liposomes and Nanoparticles

The use of liposomes and/or nanoparticles is also contemplated in the present invention. The formation and use of liposomes is generally known to those of skill in the art, and is also described below. Liposomes are also discussed elsewhere in this specification.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The following information may also be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

4. Dosage

An effective amount of the therapeutic or preventive agent is determined based on the intended goal, for example, prevention or reduction of inflammation secondary to administration of a lipid-nucleic acid complex to a subject. The quantity to be administered, both according to number of treatments and dose, depends on the subject to be treated, the state of the subject and the protection desired. Precise amounts of the therapeutic composition also depend on the judgment of the practitioner and are peculiar to each individual.

In certain embodiments, it may be desirable to provide a continuous supply of the therapeutic compositions to the patient. For topical administrations, repeated application would be employed. For various approaches, delayed release formulations could be used that provide limited but constant amounts of the therapeutic agent over an extended period of time. For internal application, continuous perfusion of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

5. Combination Therapy

As discussed above, the lipid-nucleic acid complexes of the present invention may include a therapeutic gene, such as a tumor suppressor gene or a gene capable of inducing apoptosis. In order to increase the effectiveness of the lipid-nucleic acid complex, it may be desirable to combine the lipid-nucleic acid complex with one or more than one of the antiinflammatory agents described elsewhere in this specification. These compositions would be provided in a combined amount effective to reduce inflammation secondary to the lipid-nucleic acid complex. This process may involve administering to the subject the lipid-nucleic acid complex and anti-inflammatory agent at the same time. This may be achieved by administering a single composition or pharmacological formulation that includes both agents, or by administering to the subject two distinct compositions or formulations, at the same time, wherein one composition includes the lipid-nucleic complex and the other includes the antiinflammatory agent or agents.

Alternatively, the lipid-nucleic acid complex may precede or follow the antiinflammatory therapy by intervals ranging from minutes to weeks. In embodiments where the agents are administered separately to the subject, one would generally ensure that a significant period of time did not expire between the time of each delivery, such that the two therapies would still be able to exert an advantageous effect on the subject. In such instances, it is contemplated that one may administer both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, lipid-nucleic acid complex is “A” and the antiinflammatory agent is “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of the lipid-nucleic acid complex and antiinflammatory agents of the present invention to a patient will follow general protocols for the administration of such agents, taking into account the toxicity, if any, of the agents. It is expected that the treatment cycles would be repeated as necessary.

It also is contemplated that various secondary forms of therapy, such as surgical intervention, chemotherapy, or radiotherapy, may be applied in combination with the described lipid-nucleic acid-antiinflammatory therapy of the present invention. For example, these secondary therapies may be applied in combination with the compositions of the present invention to treat a patient with cancer. One of ordinary skill in the art would be familiar with these secondary therapies, and would understand how to combine these therapies with the compositions of the present invention.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Effect of Naproxen on Transgene Expression In Vivo

Purpose

These in vivo studies were conducted to determine whether Naproxen can inhibit the DOTAP:Chol-DNA complex mediated transgene expression in vivo and thereby affect the therapeutic efficacy. These in vivo studies therefore utilized oral administration of Naproxen followed by intravenous DOTAP:Chol-FUS1 treatment in immunocompetent C3H mice for testing the inhibitory effects.

Materials and Methods

Animals. Female C3H mice (4-6 weeks old) were purchased from NCI (Fredericksburg, Md.) and housed in a pathogen free room in the Department of Veterinary Medicine and Surgery, M.D. Anderson Cancer Center, Houston, Tex.

Plasmid. The plasmid DNA used was pLJ-143, and the gene was FUS1. The plasmid concentration used was 10.20 mg/ml. The plasmid purification was proprietary, and the source of the plasmid DNA was Selective Genetics. All DNA preparations were stored at −70° C. Plasmid DNA used in the present study was taken out from −70° C. and used fresh.

Liposome Preparation. DOTAP and cholesterol were purchased from Avanti Lipids. DOTAP:Chol liposomes were prepared using the following procedure. The cationic lipid (DOTAP) was mixed with the neutral lipid (Chol) at equimolar concentrations. The mixed powdered lipids were dissolved in HPLC-grade chloroform (Mallinckrodt, Chesterfield, Mo.) in a 1-L round-bottomed flask. Thereafter, the clear solution was rotated on a Buchi rotary evaporator at 30° C. for 30 min to make a thin film. The flask containing the thin lipid film was dried under a vacuum for 15 min. The film was hydrated in 5% dextrose in water (D5W) to give a final concentration of 20 mM DOTAP and 20 mM cholesterol, referred to as 20 mM DOTAP:Chol. The hydrated lipid film was rotated in a water bath at 50° C. for 45 min and then at 35° C. for 10 min. The mixture was allowed to stand in the parafilm-covered flask at room temperature overnight, after which the mixture was sonicated at low frequency (Lab-Line, TranSonic 820/H) for 5 min at 50° C., transferred to a tube, and heated for 10 min at 50° C. The mixture was sequentially extruded through Whatman (Kent, UK) filters of decreasing size: 1.0, 0.45, 0.2 and 0.1 μm using syringes. Whatman Anotop filters, 0.2 um and 0.1 um, were used. Liposomes were stored under argon gas at 4° C.

Preparation of DOTAP:Cholesterol-FUS1 complex. Plasmid DNA (150 μg DNA) was diluted in D5W, and stock liposomes were diluted in D5W. Equal volumes of both the DNA solution and the liposome solution were mixed to give a final concentration of 150 μg DNA/300 μl volume (2.5 μg/5 μl). Dilution and mixing were performed in 1.5 ml Eppendorf tubes with all reagents at room temperature. The DNA solution was added rapidly at the surface of the liposome solution by using a Pipetman pipet tip. The DNA:liposome mixture was mixed rapidly up and down twice in the pipette tip. The freshly prepared complexes were used on the same day for injecting into animals.

Particle size analysis of DOTAP:Cholesterol-Fus 1 complex. The particle size of the DOTAP:Chol-FUS1 complex was determined using the N4-Coulter Particle Size analyzer (Beckman-Coulter). Briefly, 5 μl of the freshly prepared was diluted in 1 ml of water and particle size was determined using techniques known to those of ordinary skill in the art.

Spectrophotometric reading of DOTAP:Cholesterol-FUS1 complex at O.D. 400 nm. The optical density (OD) of the complex was determined using the Beckman-DU400 spectrophotometer. Briefly, 5 μl of the sample was diluted in 95 μl of D5W to make a final volume of 100 μl. The OD was determined at 400 nm. A sample with OD400 between 0.7 and 0.85 was used.

Naproxen. Clinical grade naproxen was purchased from the pharmacy of M.D. Anderson Cancer Center.

In vivo treatment of immunocompetent animals by intravenous injection of DOTAP:Cholesterol-FUS1 complex. Female C3H mice were divided into 3 groups (n=12/group). Group 1 was untreated; Group 2, received DOTAP:Chol-FUS1 complex; Group 3, received clinical grade naproxen orally 2 hour prior to receiving an injection of DOTAP:Chol-FUS1 complex. Naproxen was obtained from the pharmacy of M.D. Anderson Cancer Center. The naproxen concentrations given was 15 mg/kg. Two hours after administration of naproxen, animals in groups 2 and 3 received DOTAP:Chol-FUS1 complex intravenously via tail vein. Animals were euthanized at 2, 4, 6, and 15 h after injection of DOTAP:Chol-FUS1 complex, blood was then collected, and then the lungs harvested and snap-frozen in −80° C. Lung tissues were later homogenized, and total protein lysates prepared. Protein concentration in the lysate were determined by the BCA method and analyzed for FUS1 expression by western blot analysis. FUS1 protein was detected using a anti-FUS1 antibody. Untreated animals served as controls.

Results

Naproxen does not affect transgene expression in vivo. Analysis for FUS1 protein expression in the lungs of mice from groups 1, 2, and 3 demonstrated FUS1 protein expression only in groups 2 and 3. No significant differences were observed in the FUS1 protein expression between groups 2 and 3, suggesting that naproxen does not affect transgene expression in vivo.

### Example 2

Analysis of Naproxen Levels in Blood

Purpose

These in vivo studies were conducted to determine the circulating levels of naproxen in the blood. These in vivo studies therefore utilized oral administration of naproxen in immunocompetent C3H mice.

Materials and Methods

Animals. Female C3H mice (4-6 weeks old) were purchased from the National Cancer Institute (Fredericksburg, Md.) and housed in a pathogen-free room in the Department of Veterinary Medicine and Surgery, M.D. Anderson Cancer Center.

Naproxen. Clinical grade naproxen was purchased from the pharmacy at M.D. Anderson Cancer Center.

Administration and analysis of Naproxen. Female C3H mice (n=12) were administered clinical grade naproxen orally in a volume of 100 μl to give a final concentration of 15 mg/kg. Animals were euthanized at 2, 4, 6, and 15 hours after treatment, and blood was collected. Blood samples were stored at −80° C. until all samples were collected. They were then submitted to the Drug Analysis Core Facility at M.D. Anderson Cancer Center. Naproxen in the blood samples was detected by mass-spectrophotometric analysis.

Results

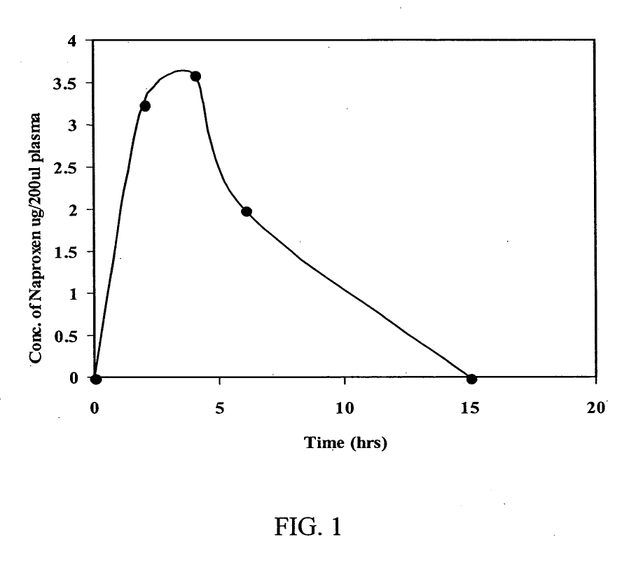

Analysis of naproxen in the blood of mice demonstrated that maximum levels (3.5 μg/200 μl of blood) of naproxen was detectable between 3 and 4 hours after treatment (FIG. 1). The levels of naproxen decreased over time and reached baseline levels by 15 hours after treatment. Blood from animals (n=3) that did not receive naproxen served as baselined controls in this experiment.

### Example 3

### DOTAP:Chol-FUS1 Complex Suppresses Cytokine Production In Vivo

Purpose

These in vivo studies were conducted to determine whether naproxen can inhibit the DOTAP:Chol-DNA complex induced inflammatory response and thereby protect mice from toxicity. These in vivo studies therefore utilized oral administration of naproxen followed by intravenous DOTAP:Chol-FUS1 treatment in immunocompetent C3H mice for testing the protective effect.

Materials and Methods

Materials and methods pertaining to the animals, plasmid, liposome preparations, preparation of the DOTAP:Cholesterol-FUS1 complex are as described in Example 1.

Particle size analysis of DOTAP:Cholesterol-Fus 1 complex. The particle size of the DOTAP:Chol-FUS1 complex was determined using the N4-Coulter Particle Size analyzer (Beckman-Coulter). Briefly, 5 μl of the freshly prepared was diluted in 1 ml of water and particle size determined.

Spectrophotometric reading of DOTAP:Cholesterol-FUS1 complex at O.D. 400 nm. The optical density (OD) of the complex was determined using the Beckman-DU400 spectrophotometer. Briefly, 5 μl of the sample was diluted in 95 μl of D5W to make a final volume of 100 μl. The OD was determined at 400 nm. A sample with OD400 between 0.7 and 0.85 was used.

In vivo treatment of immunocompetent animals by intravenous injection of DOTAP:Cholesterol-FUS1 complex. Female C3H mice were divided into 2 groups (n=15/group). Group 1 animals received naproxen at a final concentration of 15 mg/kg. Group 2 animals did not receive naproxen. 2 hour after naproxen treatment animals in both groups received an injection of DOTAP:Chol-FUS1 complex (100 μg DNA). Complexes were injected intravenously via the tail vein using a total volume of 200 μl. Animals that did not receive any treatment served as baseline controls. Blood from animals in all groups were collected at 2, 4, 6, and 15 h after injection of liposome-DNA complex, and analyzed for proinflammatory cytokines (TNF, IL-1, IL-6, IFN-γ) by ELISA (R&D Systems).

Results

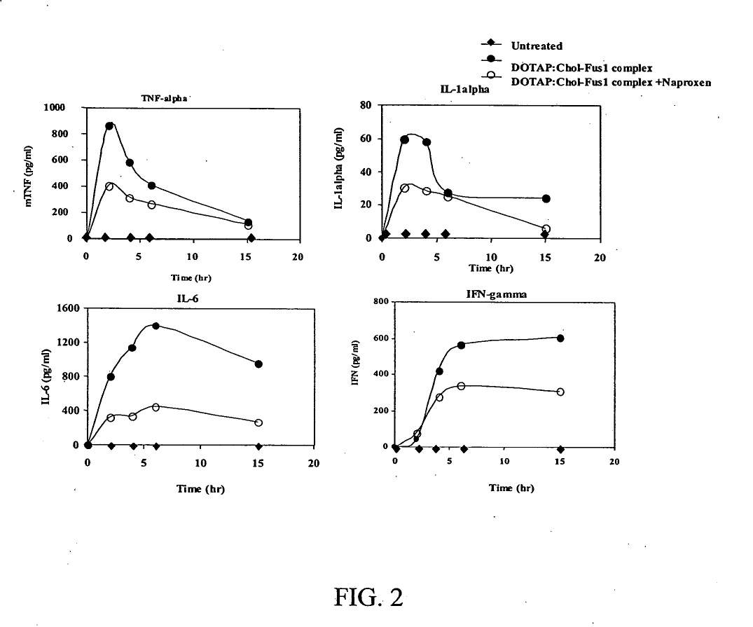

Naproxen suppresses cytokine production induced by DOTAP:Chol.—FUS1 complex. Analyses of serum samples from animals that received DOTAP:Chol-FUS1 complex demonstrated proinflammatory cytokine production (FIG. 2). TNF-alpha and IL-1 production were observed to peak at 2 hours and decreased at later time points. IL-6 and IFN-γ production were observed starting from 2 hours with maximum levels occurring at 6 hours and 15 hours, respectively. Analyses of cytokine levels in animals that had received Naproxen prior to treatment with DOTAP:Chol-FUS1 complex demonstrated 50% reduction in all of the cytokine levels. These results demonstrate that treatment of immunocompetent mice with Naproxen prior to treatment with DOTAP:Chol-FUS1 complex resulted in significant suppression of proinflammatory cytokine production.

### Example 4

### Use of Naproxen in Suppressing DOTAP:Chol-FUS1 Complex Induced Inflammatory Response

Purpose

These in vivo studies were conducted to determine whether naproxen can inhibit the DOTAP:Chol-DNA complex induced inflammatory response and thereby protect mice from toxicity. These in vivo studies therefore utilized oral administration of naproxen followed by intravenous DOTAP:Chol-FUS1 treatment in immunocompetent C3H mice for testing the protective effect.

Materials and Methods

Materials and methods pertaining to the animals, plasmid, liposome preparation, and preparation of DOTAP:Cholesterol-FUS1 complex are as described in Example 1.

Particle size analysis of DOTAP:Cholesterol-Fus 1 complex. The particle size of the DOTAP:Chol-FUS1 complex was determined using the N4-Coulter Particle Size analyzer (Beckman-Coulter). Briefly, 5 μl of the freshly prepared was diluted in 1 ml of water and particle size determined.

Spectrophotometric reading of DOTAP:Cholesterol-FUS1 complex at O.D. 400 nm. The optical density (OD) of the complex was determined using the Beckman-DU400 spectrophotometer. Briefly, 5 μl of the sample was diluted in 95 μl of D5W to make a final volume of 100 μl. The OD was determined at 400 nm. A sample with OD400 between 0.7 and 0.85 was used.

In vivo treatment of immunocompetent animals by intravenous injection of DOTAP:Cholesterol-FUS1 complex. Female C3H mice were divided into 3 groups (n=5/group). Group 1, and Group 2 received clinical grade naproxen orally 2 hour prior to receiving an injection of DOTAP:Chol-FUS1 complex. Naproxen was obtained from the pharmacy of M.D.Anderson Cancer Center. The naproxen concentrations given were 5 mg/kg and 15 mg/kg to Group 1 and Group 2 animals respectively. Two-hours after administration of naproxen, animals in all 3 groups received DOTAP:Chol-FUS1 complex (100 μg DNA). Complexes were injected intravenously via tail vein in a total volume of 200 μl. Animals were monitored initially at 1, 2, 6, 24, 48 hr and later every day until day 14. Animals that appeared morbid or moribund were euthanized by CO₂ inhalation and various organs (lung, liver, kidneys, spleen, heart) were collected in buffered formalin and submitted for histopathological examination. Organs were also collected from animals that were dead, and these were submitted for histopathological analyses.

Results

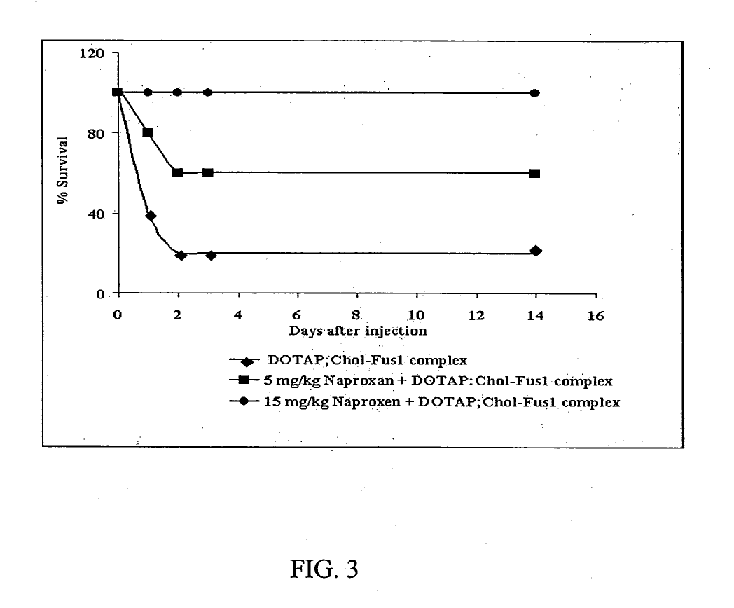

Naproxen protects mice from DOTAPC:Chol.-FUS1 complex induced toxicity. Animals in Groups 1 and 2 that were treated with naproxen were protected from DOTAP:Chol-FUS1 DNA complex induced toxicity compared to animals from Group 3 (FIG. 3). The protection offered by naproxen was dose-dependent with 60% protection observed in Group 1 and 100% protection observed in group 2 (FIG. 3). Animals that were protected remained alive on day 14. In contrast, 80% of the animals in group 3 were dead within 48 hours. Histopathological analyses of the tissues from animals that received Naproxen and those that did not are shown in Table 5. These results demonstrate that treatment of immunocompetent mice with naproxen prior to treatment with DOTAP:Chol-FUS1 complex resulted in protection and survival of mice compared to animals that did not receive naproxen.

TABLE 5

| | DIAGNOSIS |
|---|---|
| Control | No Significant Microscopic Lesion to any organs |
| Lipsome: DNA (Fus1) | No Significant Microscopic Lesions-lung, heart, |
| complex, 2 hr. | No Significant Microscopic Lesions-liver, spleen |
| Naproxen + Lipsome: | No Significant Microscopic Lesions-lung, heart |
| DNA (Fus1) complex, | No Significant Microscopic Lesions-liver |
| 2 hr | No Significant Microscopic Lesions-spleen |
| Lipsome: DNA (Fus1 | Focal acute pneumonitis and pulmonary |
| complex, 15 hr | Edema lung, Mild lymphoid atrophy spleen, |
| | Mild multifocal acute necrosis liver |
| Naproxen + Lipsome: | Moderate lymphoid atrophy spleen |
| DNA (Fus1) complex, | |
| 15 hr | |

### Example 5

### DOTAP:Chol-FUS1 Complex Induces NF-κB Expression in Lung Tumor Cells

Purpose

In vitro pilot studies were conducted to determine whether DOTAP:Chol-DNA complex induced NFkB expression in vitro.

Materials and Methods

Cells. Human lung tumor cells (A549) were grown in Hams/F12 medium and maintained in 5% CO₂ incubator.

Plasmid. The plasmid used was pLJ-143 containing FUS1 gene. The plasmid concentration was 10.20 mg/ml; purification was proprietary, and the source of the plasmid was Selective Genetics. All DNA preparations were stored at −70° C. Plasmid DNA used in the present study was taken out from −70° C. and used fresh.

Liposome Preparation. DOTAP and cholesterol were purchased from Avanti Lipids. DOTAP:Chol liposomes were prepared using the following procedure. The cationic lipid (DOTAP) was mixed with the neutral lipid (Chol) at equimolar concentrations. The mixed powdered lipids were dissolved in HPLC-grade chloroform (Mallinckrodt, Chesterfield, Mo.) in a 1-L round-bottomed flask. Thereafter, the clear solution was rotated on a Buchi rotary evaporator at 30° C. for 30 min to make a thin film, and the flask containing the thin lipid film was dried under vacuum for 15 min. The film was hydrated in 5% dextrose in water (D5W) to give a final concentration of 20 mM DOTAP and 20 mM cholesterol, referred to as 20 mM DOTAP:Chol. The hydrated lipid film was rotated in a water bath at 50° C. for 45 min and then at 35° C. for 10 min. The mixture was allowed to stand in the parafilm—covered flask at room temperature overnight, after which the mixture was sonicated at low frequency (Lab-Line, TranSonic 820/H) for 5 min at 50° C., transferred to a tube, and heated for 10 min at 50° C. The mixture was sequentially extruded through Whatman (Kent, UK) filters of decreasing size: 1.0, 0.45, 0.2 and 0.1 μm using syringes. Whatman Anotop filters, 0.2 μm and 0.1 μm, were used. Liposomes are stored under argon gas at 4° C.

Preparation of DOTAP:Cholesterol-FUS1 complex. Plasmid DNA (150 μg DNA) was diluted in D5W, and stock liposomes were diluted in D5W. Equal volumes of both the DNA solution and the liposome solution were mixed to give a final concentration of 150 μg DNA/300 μl volume (2.5 μg/5 μl). Dilution and mixing were performed in 1.5 ml Eppendorf tubes with all reagents at room temperature. The DNA solution was added rapidly at the surface of the liposome solution by using a Pipetman pipet tip. The DNA:liposome mixture was mixed rapidly up and down twice in the pipette tip. The freshly prepared complexes were used on the same day for injecting into animals.

Particle size analysis of DOTAP:Cholesterol-Fus 1 complex. The particle size of the DOTAP:Chol-FUS1 complex was determined using the N4-Coulter Particle Size analyzer (Beckman-Coulter). Briefly, 5 μl of the freshly prepared complex was diluted in 1 ml of water and particle size determined.

Spectrophotometric reading of DOTAP:Cholesterol-FUS1 complex at O.D. 400 nm. The optical density (OD) of the complex was determined using the Beckman-DU400 spectrophotometer. Briefly, 5 μl of the sample was diluted in 95 μl of D5W to make a final volume of 100 μl. The OD was determined at 400 nm. A sample with OD400 between 0.7 and 0.85 was used.

In vitro treatment of A549 cells with DOTAP:Cholesterol-FUS1 complex. Cells were seeded in 6-well plates. 24 h after incubation cells were treated as follows: no treatment, treatment with DOTAP:Chol-FUS1 complex (2.5 μg DNA), and treatment with human recombinant IL-1 alpha (100 ng/ml). Cells were harvested, homogenized, and total protein lysates prepared. Protein concentration in the lysate were determined by BCA method and analyzed for NFkB expression by western blot analysis as previously described. NFkB protein was detected using a anti-NFkB antibody.

Results

Particle size determination and OD 400 of DOTAP:Chol-Fus 1 complex. Results of the particle size analysis of the DOTAP:Chol FUS1 complex showed that the size of the complex varied between 374-400 nm.

DOTAP:Chol-FUS1 complex activates NFkB expression induced in vitro. Analysis for NFkB protein expression in A549 cells demonstrated DOTAP:Chol-FUS1 complex induced NFkB expression at levels similar to IL-1 alpha. Induction of NFkB expression by DOTAP:Chol-FUS1 was higher than in untreated control cells. Beta actin was used as internal control in these experiments. These studies indicate that treatment of A549 lung tumor cells with DOTAP:Chol-FUS1 complex induces NFkB expression.

### Example 6

### Cyclosporin A Inhibits the DOTAP:Chol-FUS1 Complex Mediated NF-κB Expression In Vitro

Purpose

These in vitro studies were conducted to determine whether cyclosporin A can inhibit the DOTAP:Chol-DNA complex mediated NFkB expression in vitro.

Materials and Methods

Materials and methods pertaining to cells, plasmid, liposome preparation, preparation of DOTAP:Chol-FUS1 complex, particle size analysis, and spectrophotometric readings of DOTAP:Chol FUS1 complex are the same as in Example 5.

In vitro treatment of A549 cells with DOTAP:Chol FUS1 complex. Cells were seeded in 6-well plates. 24 h after incubation, cells were treated with different doses of cyclosporin A (2.5 μM, 10 μM, and 50 μM). 2 h later, cells were treated as follows: no treatment, treatment with DOTAP:Chol FUS1 complex (2.5 μg DNA), and treatment with human recombinant IL-1 alpha (100 ng/ml). Cells were harvested, homogenized, and total protein lysates prepared. Protein concentration in the lysate was determined using the BCA method and analyzed for NFκB expression by western blot analysis. NFκB protein was detected using an anti-NFκB antibody.

Results

Particle size determination and OD 400 of DOTAP:Chol-Fus 1 complex. Same as Example 5.

Cyclosporin treatment inhibits NFkB expression induced by DOTAP:Chol-FUS1 complex in vitro. Analysis for NFkB protein expression in A549 cells demonstrated DOTAP:Chol-FUS1 complex induced NFkB expression at levels similar to IL-1 alpha. However, in the presence of cylosporin A, activation of NFkB was inhibited. Inhibition of NFkB was observed to occur in a dose-dependent manner with maximum inhibition occurring with 50 μM cyclosporin. Activation of NFkB by IL-1 alpha was also inhibited by cyclosporin. Beta actin was used as internal control in these experiments. These results demonstrate that treatment of A549 lung tumor cells with cyclosporin A prior to treatment with DOTAP:Chol-FUS1 complex inhibits NFkB expression.

### Example 7

### Cyclosporin A Protects Mice From DOTAP:Chol-FUS1 Complex Induced Toxicity In Vivo

Purpose

These in vivo studies were conducted to determine whether Cyclosporin A can inhibit the DOTAP:Chol-FUS1 DNA complex induced toxicity. These in vivo studies therefore utilized oral administration of cyclosporin followed by intravenous DOTAP:Chol-FUS1 treatment in immunocompetent C3H mice for testing the protective effect.

Materials and Methods

Animals. Female C3H mice (4-6 weeks old) were purchased from National Cancer Institute, (Frederick, Md.) and housed in pathogen free room in the Department of Veterinary Medicine and Surgery, M.D. Anderson Cancer Center.

Materials and Methods pertaining to plasmid, liposome preparation, preparation of DOTAP:Chol-FUS1 complex, particle size analysis, and spectrophotometric reading of DOTAP:Chol-FUS1 complex were the same as in Example 5.

In vivo treatment of immunocompetent animals by intravenous injection of DOTAP:Cholesterol-FUS1 complex. Female C3H mice were divided into 2 groups (n=5/group). Cyclosporin was given orally to animals in Group 2 (100 mg/Kg). 24 hour after cyclosporine administration, an injection of DOTAP:Chol-FUS1 complex (100 μg DNA) was given to animals in Groups 1, and 2. Complexes were injected intravenously via tail vein in a total volume of 200 μl. Animals were monitored for toxicity initially at 1, 2, 6, 24, 48, 72 hr and on day 14.

Results

Particle size determination and OD 400 of DOTAP:Chol-Fus 1 complex. Same as in Example 5.

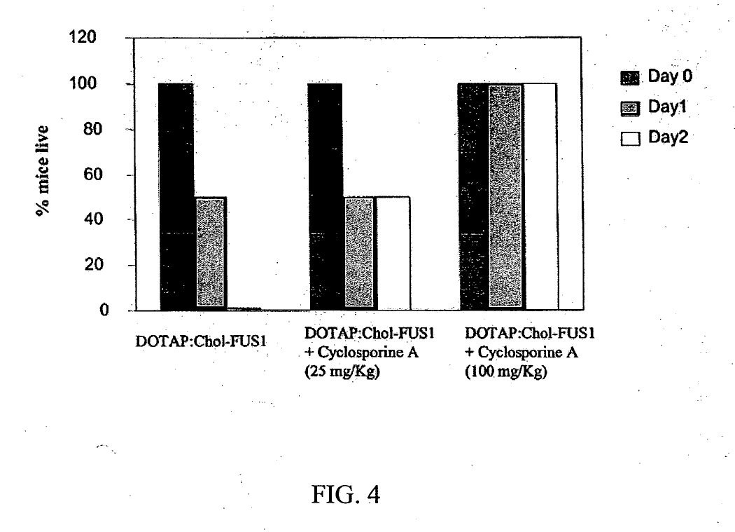

Cyclosporin protects mice from DOTAPC:Chol-FUS1 complex induced toxicity. Animals in Group 2 that were treated with cyclosporin were protected from DOTAP:Chol-FUS1 DNA complex induced toxicity compared to animals from Group 1 (FIG. 4). The protection offered by cyclosporin was 100% protection up to 48 h compared to animals in Group 1. However, cyclosporin treatment protected 60% of the animals as indicated by their survival on day 14 after treatment. All the animals from group 1 died within 72 h. These results indicate that treatment of immunocompetent mice with Cyclosporin prior to treatment with DOTAP:Chol-FUS1 complex resulted in protection and survival of mice compared to animals that did not receive cyclosporin.

### Example 8

### Cyclosporin A Can Inhibit DOTAP:Chol-FUS1 Complex Induced Toxicity In Vivo Following Oral Administration of Cyclosporin Followed by Intravenous DOTAP:Chol-FUS1 Treatment

Purpose

These in vivo studies were conducted to determine whether Cyclosporin can inhibit the DOTAP:Chol-FUS1 DNA complex induced toxicity. These in vivo studies therefore utilized oral administration of cyclosporin followed by intravenous DOTAP:Chol-FUS1 treatment in immunocompetent C3H mice for testing the protective effect.

Materials and Methods

Regarding the Materials and Methods, animals, plasmids, liposome preparation, preparation of DOTAP:Cholesterol-FUS1 complex, particle size analysis of DOTAP:Cholesterol-Fus 1 complex, and the spectrophotometric reading of DOTAP:Cholesterol-FUS1 complex at O.D. 400 nm were the same as in Example 6.

In vivo treatment of immunocompetent animals by intravenous injection of DOTAP:Cholesterol-FUS1 complex. Female C3H mice were divided into 3 groups (n=3/group). Cyclosporin was given orally to animals in Group 2 (25 mg/kg), and to animals in Group 3 (100 mg/kg). 24 hour after cyclosporine administration, a tail vein injection of DOTAP:Chol-FUS1 complex (70 μg DNA) was given to animals in Groups 1, 2, and 3. Complexes were injected intravenously via tail vein in a total volume of 200 μl. Animals were monitored for toxicity initially at 1, 2, 6, 24, and 48 hr.

Results

Particle size determination and OD 400 of DOTAP:Chol-Fus 1 complex. Same as in Example 6.

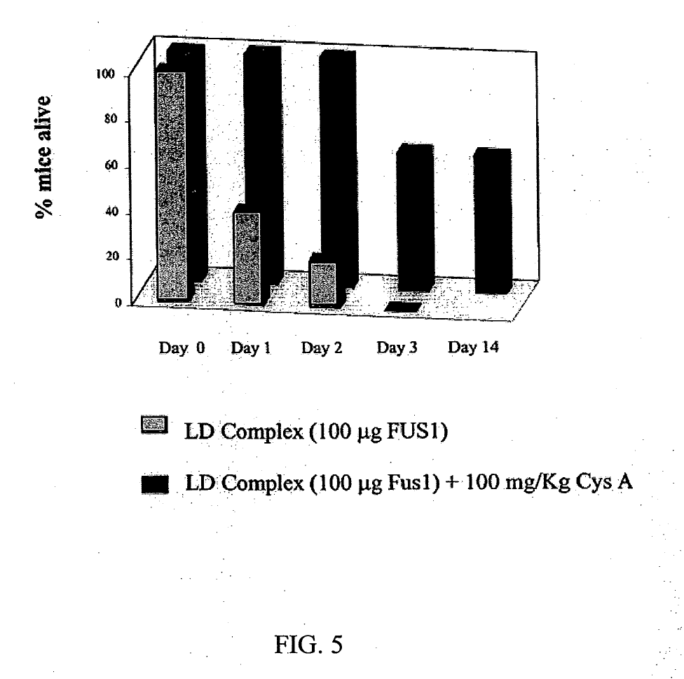

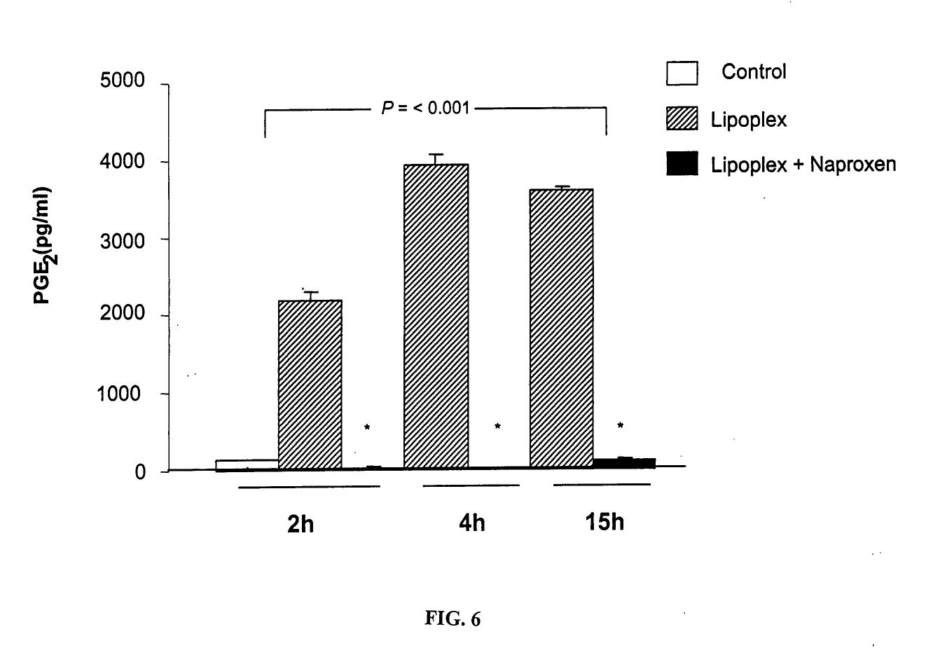

Cyclosporin protects mice from DOTAPC:Chol.-FUS1 complex induced toxicity. Animals in Groups 2 and 3 that were treated with cyclosporin were protected from DOTAP:Chol-FUS1 DNA complex induced toxicity compared to animals from Group 1 (FIG. 5). The protection offered by cyclosporin was dose-dependent with 100% protection observed in Group 3 animals that received 100 mg/kg cyclosporin. Animals in group 1 died within 48 h. These results demonstrate that treatment of immunocompetent mice with cyclosporin A prior to treatment with DOTAP:Chol-FUS1 complex resulted in a dose-dependent protection and survival of mice compared to animals that did not receive cyclosporin A.

### Example 9

### Nanoparticle Based Systemic Gene Therapy For Lung Cancer: Molecular Mechanisms, and Strategies To Suppress Nanoparticle-Mediated Inflammatory Response

Materials and Methods

Materials. All lipids (DOTAP, Cholesterol) were purchased from Avanti Polar Lipids (Alabaster, Ala.). Naproxen for tissue culture experiments was purchased from Sigma Chemicals (St. Louis, Mo.). Clinical grade Naproxen for in vivo studies was purchased from the Pharmacy at M. D. Anderson Cancer Center (Houston, Tex.). U0126 and SB203580 were purchased from Calbiochem (San Diego, Calif.). Antibodies against phosphorylated p38MAPK, pJNK, p44/42MAPK, pATF₂, and pc-Jun were purchased from Cell Signaling (Cambridge, Mass.). Anti-COX-2 antibody was purchased from Cayman Chemicals (Ann Arbor, Mich.).

Cells and culture methods. Human fibroblast (MRC-9) cell line was purchased from American Tissue Culture Collection (Rockville, Md.). Cells were maintained in the appropriate medium as recommended by the supplier. Cells were regularly passaged and maintained at 37° C. in humidified atmosphere with 5% CO₂.

Animals. Four- to six-week-old female C3H/Ncr mice (National Cancer Institute, Frederick, Md.) used in the study were maintained in a pathogen-free environment and handled according to institutional guidelines established for animal care and use.

Preparation of DNA-nanoparticles. Synthesis, and preparation of DOTAP:Chol. nanoparticles carrying the FUS1 gene (FUS1-nanoparticles) was carried out as previously described (Ramesh et al., 2001a). Freshly prepared FUS1-nanoparticles were used in each experiment described in the present study. Particle size analysis showed the FUS1-nanoparticles were 300-350 nm in size.

In vitro experiments. To determine the effect of FUS1-nanoparticles on the activation of signaling molecules associated with inflammation, MRC-9 fibroblast cells were seeded in six-well plates (5×10⁵ cells/well) and incubated overnight at 37° C. and 5% CO₂. The following day, tissue culture medium was replaced with fresh medium and cells were either nor treated or treated with various concentrations of SB203580 (p38MAPK inhibitor; 10, and 30 μM), U0126 (p44/42 MAPK inhibitor; 10, 30 μM), or with Naproxen (COX-2 inhibitor; 0.5 mM). Two-three hours after treatment, cells were transfected with FUS1-nanoparticles (2.5 μg DNA) in 0.2% serum medium. Cells were harvested at different time-points (2 h, 4 h, 15 h) after transfection, washed, and cell lysates prepared as previously described (Ramesh et al., 2001a). Untransfected cells treated with PBS served as control in these experiments. Cell lysates were subjected to western blotting analysis and probed with various antibodies as previously described (Ramesh et al., 2001b; Ito et al., 2003). In all the experiments, β-actin was detected using anti-β-actin antibody (Sigma Chemicals, St. Louis, Mo.) as a measure of internal loading control.

To determine the effect of inhibitors on transgene expression, cells were transfected with a marker gene, luciferase (luc), complexed with DOTAP:Chol. nanoparticles (luc-nanopartilces). All other experimental conditions were the same as described above. Luciferase expression was determined using the luciferase assay kit (Promega, Madison, Wis.) as previously described (Ramesh et al., 2001b). Luciferase expression was expressed as relative light units per mg of protein (RLU/mg). Assays were performed in triplicates. Experiments were performed two times and the results represented as the average of two separate experiments.

Electrophoretic mobility shift assay (EMSA). MRC-9 cells were seeded in six well plates at 1.3×10⁶ cells/well for EMSA. The following day cells were replaced with 0.2% serum medium and then preincubated for 3½ hrs in the absence or presence of naproxen before the cells were transfected with FUS1-nanoparticles (2.5 μg DNA). Cells were harvested at 2, 4 and 15 h after transfection and nuclear extracts prepared. To the nuclear extracts (10 μg), DNA binding reaction mixture containing [γ-³²P]-ATP radiolabeled AP-1 oligonucleotide and 0.5 μg poly (dI-dC) were added and incubated at 25° C. for 30 min in 5× gel shift binding buffer [20% glycerol, 5 mM MgCl₂ 2.5 mM EDTA, 2.5 mM DTT, 250 mM NaCl, 50 mM Tris-HCl (pH 7.5)]. The complexes were subsequently resolved on 5% nondenaturing polyacrylamide gels in 0.5×Tris-borate EDTA buffer for 1 h 30 min at 300 V. The bands were visualized by autoradiography.

PGE₂ production assay. Cells were seeded in 6-well plates (1-3×10⁶ cells/well) and incubated at 37° C. Twenty-four hours later, the culture medium was replaced and replenished with fresh medium supplemented with 0.2% serum. Cells were then either not treated or treated with naproxen (0.5 mM). At 3.5 h after treatment cells were transfected with FUS1-nanoparticles (2.5 μg DNA). The amount of PGE₂ secreted into the culture supernatant at various time (2 h, 4 h, and 15 h) points was determined using the PGE₂ enzyme immunoassay (Cayman Chemicals, Ann Arbor, Mich.). Assay was performed according to manufacturer's protocol.

In vivo experiments. To determine the effect of intravenous administration of FUS1-nanoparticles on inflammation and the potential use of inhibitors, in vivo experiments were conducted in immunocompetent female C3H mice. Mice were divided into three groups (n=5/group). Group 1 did not receive any treatment. Group 2 received a single dose of Naproxen (15 mg/Kg) orally 3 h prior to intravenous injection of FUS1-nanoparticles. Group 3 received intravenous injection of FUS1-nanoparticles only. The amount of plasmid DNA injected was 100 μg. The rationale for selecting this dose was based on previous results which showed that 100 μg of FUS1 plasmid DNA to produce acute inflammation that was lethal producing 100% mortality. The procedure for intravenous injections of DNA containing nanoparticles has previously been reported (Ramesh et al., 2001a; Ito et al.,. 2003). At 2 h, 4 h, 6 h and 15 h after treatment with FUS1-nanoparticles, animals were euthanized and blood and organs (lung, liver, spleen) collected. Blood samples were analyzed for mouse TNF-α by ELISA (R&D Systems, MI). Tissue samples were analyzed for expression of inflammation-associated signaling molecules by western blotting (Ito et al., 2003).

Results

FUS1-nanoparticles induces inflammation-associated signaling molecules in vitro. To test whether FUS1-nanoparticles can induce inflammation-associated signaling molecules and whose expression small molecule inhibitors, can suppress, in vitro experiments were first conducted. Transfection of MRC-9 cells with FUS1-nanoparticles resulted in a significant increase in the expression of p38MAPK, pJNK, p44/42MAPK, and its downstream substrates pATF-2, pc-Jun, and COX-2 compared to untreated control cells. The activation of various inflammation-associated signaling molecules indicate the ability of FUS1-nanoparticles to induce an inflammatory response.

Small molecule inhibitors suppress inflammation-associated signaling molecules induced by FUS1-nanoparticles. The ability of FUS1-nanoparticles to induce inflammation-associated signaling molecules in vitro suggested its potential limitation in vivo. Therefore the ability of small molecule inhibitors to inhibit inflammation-associated signaling molecules induced by FUS1-nanoparticles was tested. For this purpose, inhibitors specifically targeted towards p38MAPK (SB 203580), p44/42 MAPK (U0126) or COX-2 inhibitor (Naproxen) were targeted. Treatment of cells with various doses of SB 203580 prior to transfection with FUS1-nanoparticles resulted in a marked suppression of p38 MAPK expression and its downstream substrates, pATF-2, pc-Jun, and COX-2 compared to cells that were only transfected with FUS1-nanoparticles. The inhibitory effect was observed to be time-dependent and not dose-dependent. Baseline p38MAPK expression was observed in untreated control cells. Similarly, treatment of cells with U0126 resulted in a significant inhibition in p44/42 MAPK expression and its downstream substrates compared to cells that did not receive any treatment and cells that were transfected with FUS1-nanoparticles only. The inhibitory activity exerted by U0126 was neither time-nor dose-dependent. P44/42MAPK expression levels were decreased more than the baseline expression seen in untreated control cells. These results suggest that p38MAPK and p44/42MAPK can be effectively inhibited using inhibitors targeted towards these molecules.

The effect of naproxen, a non-steroidal anti-inflammatory small molecule targeted to COX-2 was next investigated. Treatment of cells with naproxen prior to transfection with FUS1-nanoparticles resulted in a significant inhibition of various MAPK that included p38MAPK, pJNK, and p44/42MAPK compared to cells that were transfected with FUS1-nanoparticles only. The inhibitory effect on various MAPK correlated with decreased expression of their downstream substrates, pATF-2, pc-Jun and COX-2. Additionally the inhibitory effect on MAPK expression appeared to increase over time. Baseline expression of p38MAPK, pJNK, and p44/42MAPK was observed in untreated control cells. These results demonstrate that naproxen in addition to inhibiting COX-2 also inhibited all three kinases that are associated with inflammation. Thus naproxen appears to function as a broad-spectrum inhibitor inhibiting multiple signaling molecules. Furthermore, based on its ability of to function as a broad-spectrum inhibitor it was speculated that naproxen would be more effective than SB 203580 and U1026 in vivo. Hence, in all subsequent experiments, naproxen was tested.

FUS1-nanoparticle-mediated activation of AP-1 is inhibited by naproxen. Recent studies have demonstrated activation of p38MAPK by CpG containing DNA leads to the activation of transcription factor CREB/AP-1, that is an important mediator of inflammation (Yeo et al., 2003). Presence of consensus AP-1 DNA binding site in the promoter region of several genes including COX-2 has been reported (Yeo et al., 2003). Based on these reports and ability of FUS1-nanoparticles to induce COX-2 expression, it was speculated activation of AP-1 and that pretreatment with naproxen will result in reduced AP-1 DNA binding activity. Therefore to test this possibility, cells treated with FUS1-nanoparticles in the presence or absence of naproxen were analyzed for AP-1 DNA binding activity by electro-mobility shift assay (EMSA). Untreated cells served as control in these experiments. Increased AP-1 activity was observed in FUS1-nanoparticles transfected cells compared to untreated control cells. In contrast, treatment with naproxen resulted in inhibition of AP-1 activity. This data shows that FUS1-nanoparticle treatment results in activation of AP-1 that is inhibited by naproxen. Furthermore, AP-1 activation correlated with the activation of MAPK molecules that are upstream to these transcriptional factors. Correlation was also observed with the activation of COX-2 that is downstream of AP-1.

FUS1-nanoparticle induced PGE₂ production is inhibited by naproxen. PGE₂ is a substrate for COX-2. Activation of COX-2 results in breakdown of PGE₂ into its metabolites that are potent inducers of inflammation (DeWitt, 1991; Ghosh et al., 2001). Since naproxen inhibited lipoplex-induced COX-2 expression, studies were conducted to determine whether PGE₂ production is also inhibited. To test this possibility, secreted PGE₂ levels were measured in the tissue culture medium growing cells that were transfected with FUS1-nanoparticles in the presence or absence of naproxen. PGE₂ expression levels were determined by ELISA. As shown in FIG. 6, treatment of cells with FUS1-nanoparticles resulted in a time-dependent increase in the secreted PGE₂ levels (2000-4000 pg/ml) compared to the basal level in untreated control cells (10 pg/ml). However, pretreatment of cells with naproxen prior to transfection with FUS1-nanoparticles resulted in a significant inhibition of PGE₂ (33-120 pg/ml; P=<0.001). In fact the inhibition was almost complete starting from 2 h after transfection. Similar results were also obtained from murine macrophage cell line (RAW 264.7; data not shown). These results demonstrate the ability to naproxen to effectively inhibit both COX-2 expression and its substrate PGE₂ that are important mediators of inflammation.

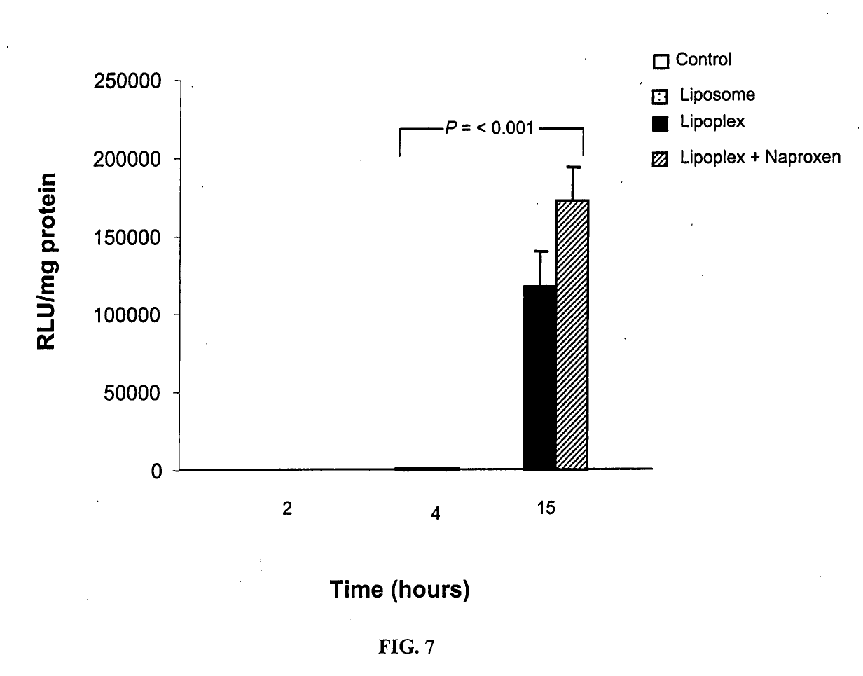

Nanoparticle-mediated transgene expression in not affected by naproxen. Although suppression of nanoparticle-mediated signaling molecules was demonstrated, one question that remains unanswered are the effects of the inhibitors on transgene expression. The possibility that the inhibitors can also suppress transgene expression existed. Furthermore, previous studies have shown that inflammatory cytokines inhibit transgene expression (Battz et al., 2001). Therefore, studies were conducted to investigate the effect of naproxen treatment on transgene expression using luciferase as a marker gene. Luciferase expression was observed at 15 h in both, cells that were transfected with luc-nanoparticles containing naproxen and in cells that were transfected with luc-nanoparticles and not treated with naproxen (FIG. 7; P=<0.001). Furthermore, luciferase expression was greatly increased in naproxen treated cells compared to cells that were not treated with naproxen. No luciferase expression was observed in cells that were untreated (control) or treated with empty nanoparticles. Enhanced transgene expression was also observed in lung tumor cells that were transfected with luc-nanoparticles in the presence of naproxen. Thus, naproxen treatment results in selective inhibition of signaling molecules associated with inflammation without affecting transgene expression.

FUS1-nanoparticles-induced inflammatory response is suppressed by naproxen in vivo. Preliminary studies demonstrated that intravenous injection of FUS1-nanoparticles resulted in the induction of an inflammatory response that was dose-dependent. Injection of 100 μg of FUS1 plasmid DNA complexed to DOTAP:Chol. nanoparticles resulted in acute inflammatory response resulting in 100% mortality. Based on these observations, studies were conducted to evaluate whether pretreatment of animals with naproxen prior to intravenous injection of a lethal dose of FUS1-nanoparticles would suppress the acute inflammatory response. Suppression of inflammation by naproxen was determined by measuring TNF-α, a key mediator of inflammation (Palladino et al., 2003), and by analyzing the lung tissues for the inflammation-associated signaling molecules at various (2 h, 4 h, 15 h) time points after treatment.

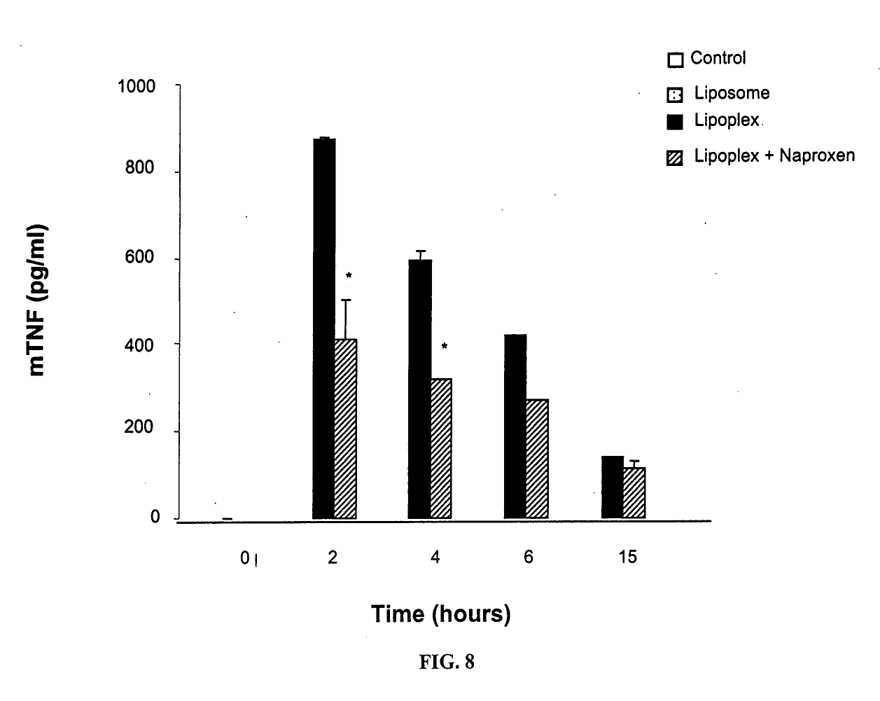

Analysis for TNF-α in the blood of animals that were injected with FUS1-nanoparticles showed maximum TNF-α expression levels at 2 h (873 pg/ml) and decreased over time (FIG. 8A; P=<0.04). In contrast, the TNF-α expression levels was reduced by half at 2 h (411 pg/ml) in animals that were pretreated with naproxen prior to injection of FUS1-nanoparticles. Reduced TNF-α in naproxen treated animals was also observed at all time points tested. These results showed that naproxen suppressed FUS1-nanoparticles induced TNF-α.

The expression of inflammation-associated signaling molecules in the lung tissues of mice that were either treated with naproxen or not treated with naproxen was next tested. As observed in the in vitro experiments, a marked activation of p38MAPK, pJNK, p44/42MAPK and their downstream substrates pATF2, pc-JUN, and COX-2 was observed in the lung of mice that were intravenously injected with FUS1-nanoparticlces compared to the lungs of control mice that did not receive any treatment. Activation of the signaling molecules was observed at all time points tested with maximum activation occurring at 2 h that correlated with TNF-α production. However, activation of the various signaling molecules was markedly suppressed in the lungs of mice that were treated with naproxen prior to receiving FUS1-nanoparticles. Suppression of activation of signaling molecules by naproxen was observed as early as 2 h after FUS1-nanoparticles treatment. The inhibitory activity of naproxen on the activation of signaling molecules correlated with its inhibitory activity on TNF-α. These results show that naproxen inhibits TNF-α production by inhibiting various signaling molecules that are associated with its induction.

### Example 10

### Protective Effect of Naproxen on FUS1-Nanoparticle Induced Toxicity

Materials and Methods

Materials. All lipids (DOTAP, Cholesterol) were purchased from Avanti Polar Lipids (Alabaster, Ala.). Naproxen for tissue culture experiments was purchased from Sigma Chemicals (St. Louis, Mo.). Clinical grade Naproxen for in vivo studies was purchased from Pharmacy at M. D. Anderson Cancer Center (Houston, Tex.). SP 60012 was purchased from Biosource. SB203580 were purchased from Calbiochem (San Diego, Calif.). Antibodies against phosphorylated p38MAPK, pJNK, p44/42MAPK, pATF₂, pc-Jun, pSTAT3ser727 and pSTAT3Tyr705 were purchased from Cell Signaling (Cambridge, Mass.). Anti-COX-2 antibody was purchased from Cayman Chemicals (Ann Arbor, Mich.).

Cells and culture methods. Mouse macrophage (RAW) cell line was obtained from American Type Culture Collection, Rockville, Md. Cells were maintained in the appropriate medium as recommended by the supplier. Cells were regularly passaged and maintained at 37° C. in humidified atmosphere with 5% CO₂.

Animals. Four-to six-week-old female immunocompetent C3H/HeNcr mice (National Cancer Institute, Frederick, Md.) used in the study were maintained in a pathogen-free environment and handled according to institutional guidelines established for animal care and use.

Synthesis of liposome and preparation of DNA:liposome mixtures. Liposomes (DOTAP:Chol) were synthesized and extruded through Whatman filters (Kent, UK) of decreasing size (1.0, 0.45, 0.2, and 0.1 μm) as described previously. DNA:liposome complexes were prepared fresh 2 to 3 h before tail vein injection in mice. Briefly, DOTAP:Chol (20 mM) stock solution and stock DNA solution diluted in 5% dextrose in water (D5W) were mixed in equal volumes to give a final concentration of 4 mM DOTAP:Chol-150 μg DNA in 300 μl final volume (ratio 1:2.6). All reagents were diluted and mixed at room temperature. Reagents were genetly mixed in a 1.5 ml Eppendorf tube by pipetting. The DNA solution was added at the surface of liposome and mixed rapidly up and down twice with the pipet tip. The DNA:liposome mixture thus prepared was precipitate free and used for all in-vivo experiments. Synthesis, and preparation of lipoplex carrying the FUS1 gene was carried out as previously described (Ramesh et al., 2001a).

Particle size analysis. Freshly prepared DNA:liposome complexes were analyzed for mean particle size using the N4 particle size analyzer (Coulter, Miami, Fla.). The average mean particle size of the DNA:liposome complexes ranged between 300-350 nm.

In Vivo Experiments

Animal toxicity. To determine the efficacy of systemic treatment, survival experiments were performed using the immunocompetent female C3H mice. Mice were divided into four groups (n=5/group). Group 1 receieved no treatment, Group 2 received DOTAP:Chol nanoparticle, Group 3 received naked FUS1 plasmid and Group 4 receieved FUS1-nanoparticle. The amount of FUS1 plasmid injected was 100 μg. The procedure for intravenous injections of liposome-DNA complex has previously been reported (Ramesh et al., 2001a; Ito et al., 2003).

Animal tolerable dose. To determine the tolerable dose to animals, mice were injected with different concentration of FUS1 complex and monitered the survival of mice.

Animal Survival Experiment. To determine the efficacy of naproxen treatment, survival experiments were performed using the immunocompetent female C3H mice. Mice were divided into three groups (n=5/group). Group 1 receieved no treatment, Group 2 received 5 mg/kg naproxen was given orally, Group 3 received 15 mg/kg naproxen was given orally. All the group were injected FUS1-nanoparticle 2 h after administered naproxen. The amount of FUS1 plasmid injected was 100 μg. The procedure for intravenous injections of liposome-DNA complex has previously been reported (Ramesh et al., 2001a; Ito et al., 2003).

Cytokine profiles, Organ toxicity and Signalling molecules in C3H mice. To determine the cytokine profile, organ toxicity, signaling molecules responsible for inflammation, experiments were performed using immunocompetent female C3H mice. Group 1 receieved no treatment, Group 2 received nanoparticle, Group 3 received naked FUS1 plasmid, Group 4 receieved FUS1-nanoparticle and Group 5 receieved 15 mg/kg Naproxen orally prior to injecting the FUS1-nanoparticle. The amount of FUS1 plasmid injected was 100 μg. Serum and organ cytokine levels were determined by ELISA for murine TNF-α and murine Il-6. At indicated times after injection, blood was obtained via aspiration after right heart puncture. The blood was allowed to stand for 4 hours at 4° C. and then was centrifuged twice at 15,000 g for 10 minutes at 4° C. The supernatants (serum) were kept at −80° C. until used. Cytokine concentrations were quantified using specific ELISA kits (R&D systems) according to manufacturer's instructions. Organs were collected at 2, 4, and 15 h and were snap-frozen in liquid nitrogen and stored at 80° C. until time of ELISA and Western blot analysis. At the time of ELISA, organs were slowly thawed on ice. The lung, liver and spleen was homogenized in 750 μl PBS containing a cocktail of protease inhibitors using a tissue homogenizer. The cytoplasmic fraction was isolated as the supernatant fraction following centrifugation at 15,000 g for 20 min at 4° C. The supernatant was used for the determination of TNF-α and IL-6 were quantitated by ELISA kits purchased from R&D Sytems and Biosource.fraction.

Organ toxicity studies. The lungs, heart, liver, spleen, kidney, ovaries were removed from FUS1-nanoparticle alone treated group and naproxen followed FUS1-nanoparticle treated group. These two groups were analyzed histopathologically for the treatment-associated toxic effects.

Western blot analysis for the Signaling molecules. At the time of western blot analysis, organs were slowly thawed on ice. The organs were then washed three times with 1× with cold PBS and then homogenized using a tissue homogenizer in resuspended in lysis buffer (62.5 mM Tris-HCl, 2% SDS, 10% glycerol, 4M urea) containing a cocktail of protease inhibitors. Whole Cell lysates were collected in an Eppendorf tube and sonicated for 30 seconds and heated in a water bath at 95° C. for 5 mins and then centrifuged at 13000 rpm for 10 min at 4° C. The supernatants were mixed with 5% 2-mercaptoethanol and stored at −80° C. Protein concentrations was determined by BCA protein assay. Aliquots of cell extracts containing 50 μg of total protein were resolved in 10% SDS-PAGE and transferred from gel to nitrocellulose membrane (Hybond-ECL; Amersham Pharmacia Biotech, UK) and then blocked for 1 h at room temperature (5% nonfat milk powder and 0.1% Tween 20 in TBS or PBS). Then the membranes were incubated with the primary antibodies, phosphospcific p38 (1:1000), phosphospecific pJNK (1:1000), phosphospecific p44/42 (1:1000), phospho ATF2 (1:1000), phospho cJun (1:1000), phospho STAT3 (1:1000) and COX-2 (1:1000). The membranes were then incubated with HRP-conjugated rabbit IgG Ab (Amersham) and the bound antibodies were visualized by enhanced chemiluminescence (Amersham; Piscataway, N.J.). The expression of α-actin was used as the loading control.

Electrophoretic mobility shift assay. Nuclear extracts of whole lung tissues were prepared as described previously in Gao et al., 2004. Briefly, frozen lungs, liver and spleen were homogenized in 0.6% (v/v) Nonidet p-40, 150 mM Nacl, 10 mM HEPES (pH 7.9), 1 mM EDTA, 0.5 mM PMSF containing a 25 times of cocktail of protease inhibitors. The homogenate was incubated on ice for 30 min and then centrifuged for 10 min at 13,000 rpm at 4° C. Proteins were extracted from the pelletted nuclei by incubation at 4° C. with 420 mM NaCl, 20 mM HEPES (pH 7.9), 1.2 mM MgCl₂, 0.2 mM EDTA, 25% (v/v) glycerol, 0.5 mM DTT, 0.5 mM PMSF contaning 25× protease inhibitor. Nuclear debris was pelletted by centrifugation at 13,000 rpm for 30 min at 4° C. and the supernatant extract was collected and stored at −80° C. Protein concentrations were determined Bio-Rad protein assay (Hercules, Calif.) using double-stranded oligonuclotides containing Stat3 consensus oligonucleotide (GATCCTTCTGGGAATTCCTAGATC-3′ (SEQ ID NO:1); Santa Cruz Biotechnology, Santa Cruz, Calif.) and NF-κB consensus oligonucleotide (AGTTGAGGGGACTTTCCCAGGC (SEQ ID NO:2); Promega, Madison, Wis.). These probes were end-labelled with [γ-³²P]ATP(3000 Ci/mmol at 10 mCi/ml; Amersham Biosciences, Sunnyvale, Calif.). DNA binding reactions were performed at room temperature in a 25-μl reaction mixture containing 6-μl of nuclear extract and 5 μl of 5× binding buffer (20% (w/v) Ficoll, 50 mM HEPES (pH 7.9), 5 mM EDTA, 5 mM DTT). The reminder of the reaction mixture contained KCl at a final concentration of 50 mM, Nonidet P-40 at a final concentration of 0.1%, 1 μg of poly (dI-dC), 200 pg of probe, bromophenol blue at a final concentration of 0.06% (w/v), and water to volume. Samples were electrophoresed through 5.5% polyacrylamide gels in 0.5×TBE at 160 V for 3 h, dried under vacuum, and exposed to X-ray film after overnight expose with hyperfilm at −80° C. The gel was then dried and subjected to autoradiography.

Nuclear extracts for NFkB of whole lung tissues were prepared as described in Gao et al., 2004 Briefly, frozen lungs were minced and incubated on ice for 30 min in 0.5 ml of ice-cold buffer A, composed of 10 mM HEPES (pH 7.9), 1.5 mM KCl, 10 mM MgCl₂, 0.5 mM DTT, 0.1% NP-40 and 0.5 mM phenylmethylsulfonyl fluoride. The minced tissue was homogenized using a Dounce homogenizer and centrifuged at 14,000 rpm at 4° C. for 10 min. The nuclear pellet obtained was suspended in 0.2 ml of buffer B [20 mM HEPES (pH 7.9), 25% glycerol, 1.5 mM MgCl₂,420 mM NaCl, 0.5 mM DTT, 0.2 mM EDTA, 0.5 mM phenymethylsulfonyl fluoride, and 4 μM leupeptin] and incubated on ice for 2 h with intermittent mixing. The suspension was then centrifuged at 14,000 rpm at 4° C. fro 30 min. The suspension was then centrifuged at 14,000 rpm at 4° C. fro 30 min. The supernatant (nuclear extract) was collected and stored at −80° C. until use. The protein concentrationm was measured by the method of Bradford with BSA as the standard. EMSA was perfored by incubating 10 μg of nuclear protein extract by procedure as described previously (TCRT paper). Briefly, Double-stranded oligonucleotides consensus NFκB (Promega) were end-labelled with [γ-³²P]-ATP using T4 polynucleotide kinase. A typical binding reaction mixture contained the labeled oligonucleotide and 1 g poly (dI-dC) and nuclear protein extracts (10 μg) were incubated at 25° C. for 10 min in 5× gel shift binding buffer [50 mM HEPES, 50% glycerol, 250 mM KCl, 0.5 mM EDTA, 12.5 mM DTT]. The complexes were resolved on nondenaturing 4% polyacrylamide gels in 0.5×Tris-borate EDTA buffer for 1 h 30 min at 300 V. The bands were visualized by autoradiography.

In vitro experiments. To determine the effect of nanoparticle-FUS1 complex on the activation of signaling molecules associated with inflammation, RAW macrophage cells were seeded in six-well plates (1×10⁶ cells/well) and incubated overnight at 37° C. and 5% CO₂. The following day, tissue culture medium was replaced with fresh 0.2% medium and incubated overnight at at 37° C. and 5% CO₂. The following day tissue culture medium was replaced with fresh 0.2% medium were either nor treated or treated with Naproxen (COX-2 inhibitor; 0.5 mM). Two-three hours after treatment, cells were transfected with FUS1-nanoparticle (2.5 μg DNA) in 0.2% serum medium. Cells were harvested at different time-points (2 h, 4 h, 15 h) after transfection, washed, and cell lysates prepared as previously described (Ramesh et al., 2001a). Untransfected cells treated with PBS served as control in these experiments. Cell lysates were subjected to western blotting analysis and probed with various antibodies as previously described (Ito et al., 2003). In all the experiments, β-actin was detected using anti-β-actin antibody (Sigma Chemicals, St. Louis, Mo.) as a measure of internal loading control.

Inflammatory cytokines production assay. To determine the effect of nanoparticle-FUS1 complex on the production of inflammatory cytokines, raw macrophage cells were seeded in six-well plates (1×10⁶ cells/well) and incubated overnight at 37° C. and 5% CO₂. The following day, tissue culture medium was replaced with fresh 0.2% medium and incubated overnight at at 37° C. and 5% CO₂. The following day tissue culture medium was replaced with fresh 0.2% medium were either nor treated or treated with Naproxen (COX-2 inhibitor; 0.5 mM). Two-three hours after treatment, cells were transfected with FUS-nanoparticle (2.5 μg DNA) in 0.2% serum medium. The amount of TNF-α, IL-6 and PGE₂ secreted into the culture supernatant at various time (2 h, 4 h, and 15 h) points was determined using the TNF-α, IL-6 ELISA kit (Biosource International, California), PGE₂ enzyme immunoassay (Cayman Chemicals, Ann Arbor, Mich.). Assay was performed according to manufacturer's protocol.

To determine the effect of inhibitors on transgene expression, cells were transfected with a marker gene, luciferase (luc), complexed with nanoparticle. All other experimental conditions were the same as described above. Luciferase expression was determined using the luciferase assay kit (Promega, Madison, Wis.) as previously described in Ito et al., 2003. Luciferase expression was expressed as relative light units per mg of protein (RLU/mg). Assays were performed in triplicates. Experiments were performed two times and the results represented as the average of two separate experiments.

Results

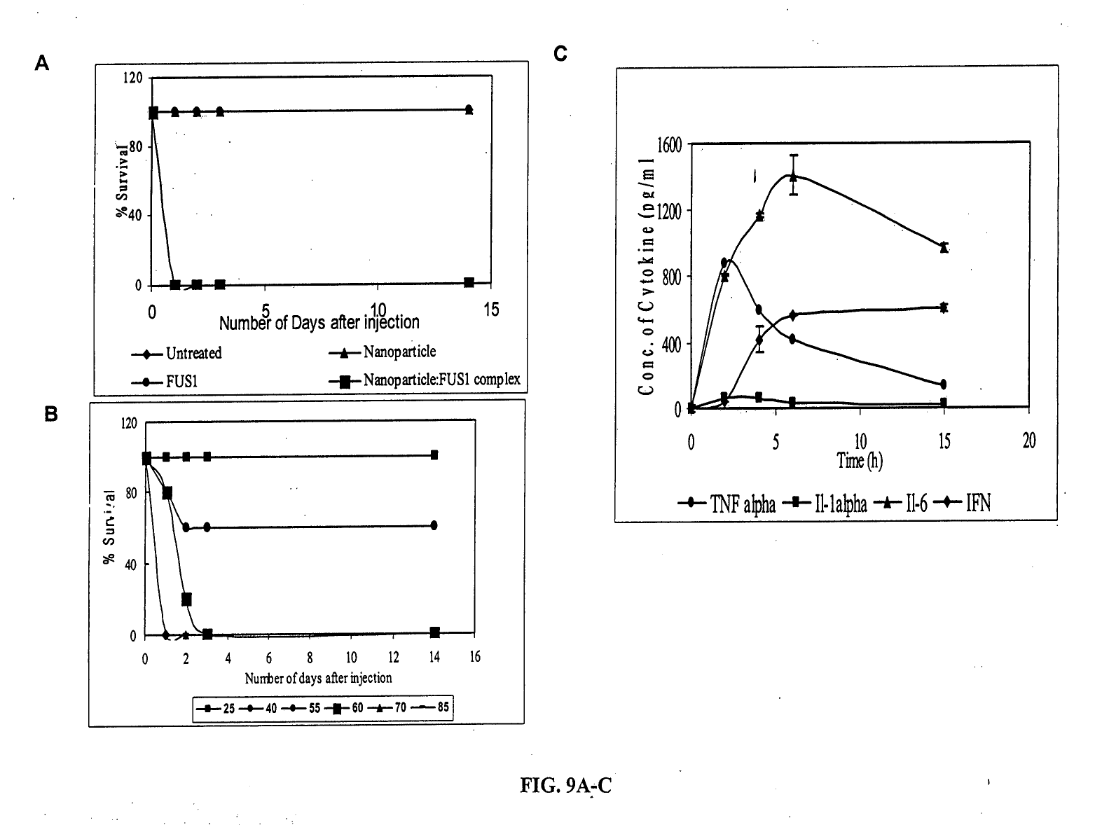

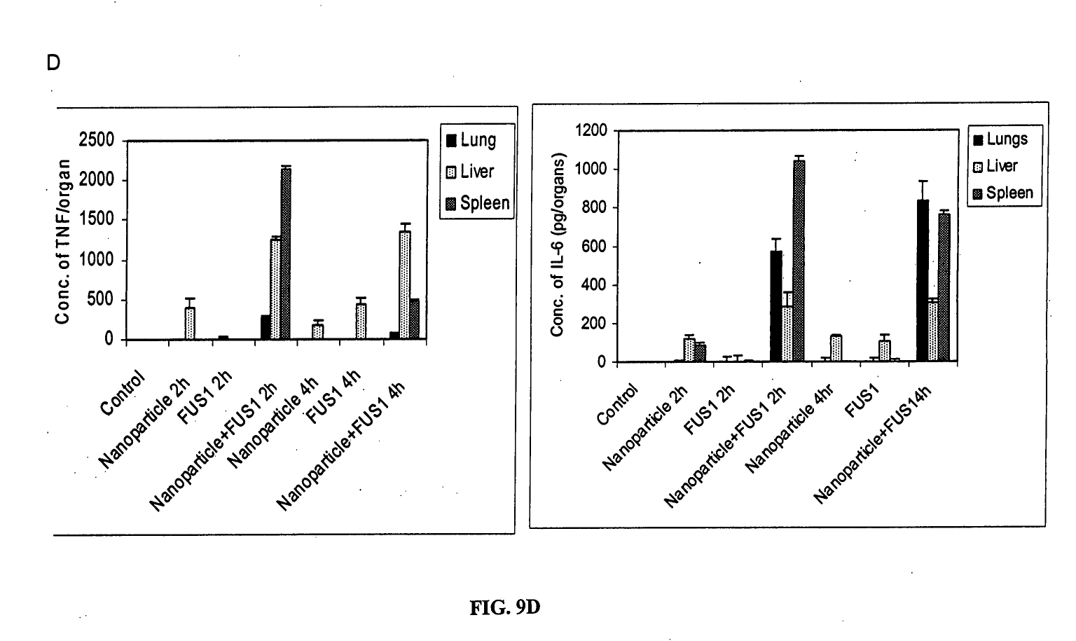

Liposome:FUS1 complex induces toxicity in the mice. FIG. 9A demonstrates that intravenous administration of 100 μg of FUS1-nanoparticle complex resulted in 100% mortality to the mice when compared to nanoparticle liposome alone, free FUS1 and untreated control. To assess the maximum tolerated dose to the mice, different concentration of FUS1 were intravenously injected, and survival of mice was monitored. As shown in FIG. 9B, 100% of mice survived at low concentration of FUS1 (25 μg and 40 μg) and 50% of the mice survived at 55 μg FUS1 when compared to 0% survival at 60, 70 and 85 μg FUS1. In all of these studies, 100 μg FUS1 was used to increase the therapeutic dose.

Next, studies were conducted to investigate the serum and organ cytokines level after intravenous injection of FUS1-nanoparticle complex. Serum and organ was collected at various times following injection and assessed for cytokine by ELISA. FIG. 9C shows the administration of FUS1-nanoparticle elicited transient rises in the serum concentrations level of TNF-α, Il-1α, Il-6 and IFN-γ. FUS1-nanoparticle induced TNF-α was detected as early as 30 min after injection, peaked at 2 h (900 pg/ml), and declined thereafter. Analysis for IL-6 in the blood of animals that were injected with FUS1-nanoparticle complex showed maxium IL-6 expression levels at 6 h (1500 pg/ml) and decreased over time. The FUS1-nanoparticle also induced rapid IFN-γ production which peaked at 6 h and remained stable until 15 h. IL-1a was very low when compared to other cytokines levels in serum.

Next, studies were conducted to investigate the cytokine profile in lungs, liver and spleen. As shown in FIG. 9D, FUS1-nanoparticle induces seceretion of TNF-α and Il-6 at 2 and also 4 h in all the organs. These results correlated well with recent studies reported by Sakurai et al., 2002.

FUS1-nanoparticle complex activation of MAPK pathways. It has previously been shown that ERK1 and ERK2, JNK/SAPK, and p38 become activated in response to CpG DNA. Studies were conducted to confirm these results. To determine whether complex upregulated MAPK activity, lung, liver and spleen were collected several time points after injecting the complex.

MAPK are activated in an FUS1-nanoparticle model of organ inflammation. It has previously been shown that ERK1 and ERK2, JNK/SAPK, and p38 become activated in response to DNA-nanoparticle. Nanoparticle-FUS1 complex was administered IV to C3H mice, and their lungs were examined for MAPK activation at time points ranging from 2 to 4 h. The time course of activation of p38, pJNK and p44/42 during FUS1-nanoparticle induced lung inflammation was evaluated by western blotting, using cell lysates from whole lung obtained at various time points after onset of lung inflammation. There was no increased expression of p38, JNK and ERK1/2 in unstimulated lungs. However, nanoparticle:FUS1 complex increases p38, JNK and ERK1/2 MAPK activation, which was evident by 2 h and became very strong by 4 h. The downstream target pATF2 and pc-Jun also increased at 2 and 4 h. Phosphorylated STAT-3 is a protein that is common to many functional STAT complexes and also involved in inflammatory response. Therefore, studies were conducted to determine, by western blot analysis, the kinetics of STAT-3 phosphorylation in response to FUS1-nanoparticle. Two phosphorylation sites, Tyr-705 and Ser-727, differentially regulate STAT-3. Because phosphorylation of both sites is required for maximal transcriptional activity, studies were conducted to determine if one or both sites are phosphorylated in response to FUS1-nanoparticle. A time-dependent phosphorylation in response to FUS1-nanoparticle was observed, and the phosphorylated band appeared 2 h following treatment with FUS1-nanoparticle, and remained elevated. It was found that STAT3 is phosphorylated on both residues Tyr-705 and Ser-727 in FUS1-nanoparticle treated lungs.

In the case of liver, the protein level of phosphorylated JNK in the FUS1-nanoparticle group was markedly enhanced at 4 h. Next, studies were conducted to investigate whether the family of STAT transcription factors were activated in response to FUS1-nanoparticle. Western blot analysis with phosphorylated STAT3 antibody suggested that expression of phosphorylated forms of tyrosine and serine STAT3 was increased at 2 and 4 h in liver after stimulation with FUS1:nanoparticles complex with those from nanoparticle and FUS1 alone group.

To determine FUS1-nanoparticle could also induces inflammatory associated signaling molecules in spleen, the spleen was harvested at different time points and analysed for various MAPK signaling proteins. FUS1-nanoparticle induced phosphorylated form STAT3 at 2 and 4 h when compared to nanoparticle, free FUS1 alone.

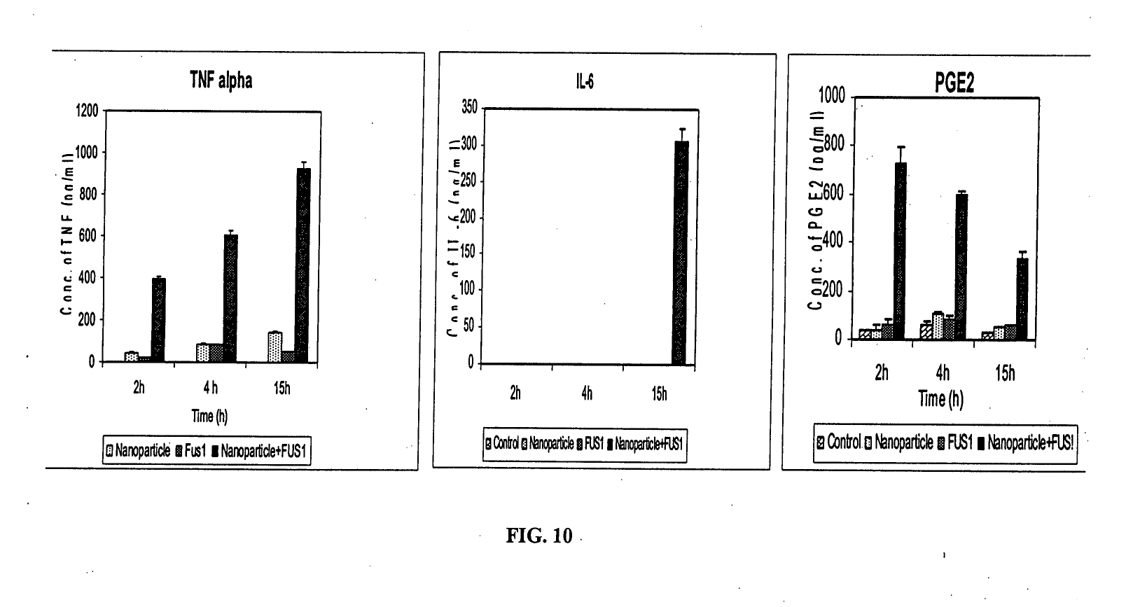

FUS1-nanoparticle induces inflammatory cytokines and signaling molecules associated with inflammation. Studies were conducted to investigate the effects of liposome:FUS1 complex on the release of TNF-α, IL-6 and PGE₂ in Raw 264.7. The murine macrophage cell line RAW 264.7 was used, which releases PGs, and pro-inflammatory cytokines such as TNF-α and IL-6 upon stimulation with CpG nucleotide, thus providing a suitable model for studying inflammatory response in cultured cells. Therefore, Raw264.7 cells were treated with medium, 4 μM nanoparticle, 2.5 μg FUS1 or FUS1-nanoparticle (2.5 μg) complex and the cell-free supernatants were collected at 2, 4 and 15 h and assayed for TNF-α, IL-6 and PGE₂. The results indicate that in murine macrophage cell line RAW 264.7, FUS1-nanoparticle stimulates TNF-α, IL-6 and PGE₂ release (FIG. 10). Upon FUS1-nanoparticle stimulation, TNF-α synthesis by murine macrophage cell line increased in a time-dependent manner. In addition, the complex increased Il-6 synthesis at 15 h compared to the nanoparticle or FUS1 alone. FUS1-nanoparticle also induces other proinflammatory cytokines, such as PGE₂, at 2 h which then decrease at later time points. Previous studies demonstrated that in RAW264.7 cells, CpG DNA induces activation of three MAPKs, ERK, JNK and p38MAPK (Kumar et al., 2003; Manning and Davis, 2003; Lai et al., 2003). To investigate whether FUS1-nanoparticle induces activation of these three MAPKs in RAW264.7 cells through the classical MAPK activation pathways, RAW264.7 cells were stimulated with medium, nanoparticle, FUS1 and FUS1-nanoparticle complex for 2 and 4 h. It was found that FUS1-nanoparticle induced phosphorlation of p38, JNK at 2 h with persistent activation at 4 h as compared to nanoparticle, FUS1 alone. FUS1-nanoparticle also induced ERK and the pcJUN, pATF-2 which is a substrate for p38 and JNK, ERK was activated at 4 h. Because phosphorylation of STAT3 could be a target for inflammation, studies were conducted to investigate whether FUS1-nanoparticle induces phosphorylation of STAT3 through an p38MAPK dependent pathway. Only at 4 h was there FUS1-nanoparticle induced phosphorylation of STAT3 at Tyr 705. These results indicate that FUS1-nanoparticle induces proinflammatory cytokines through MAPKinase pathway.

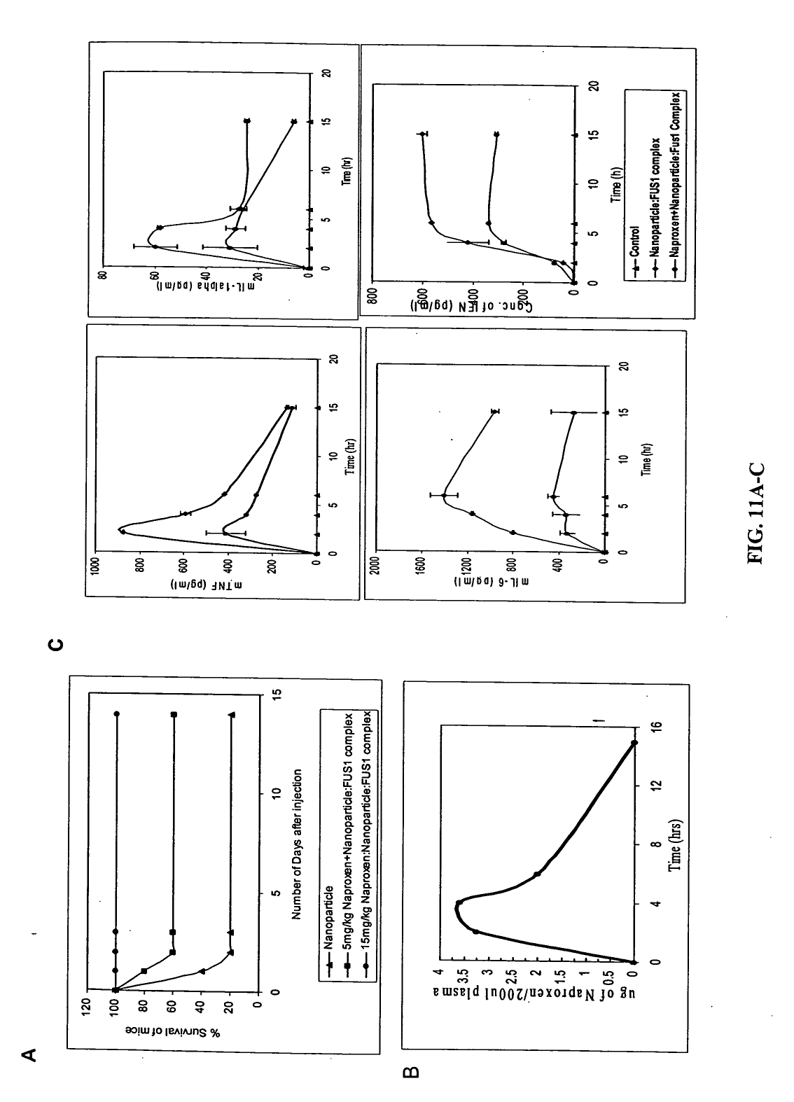

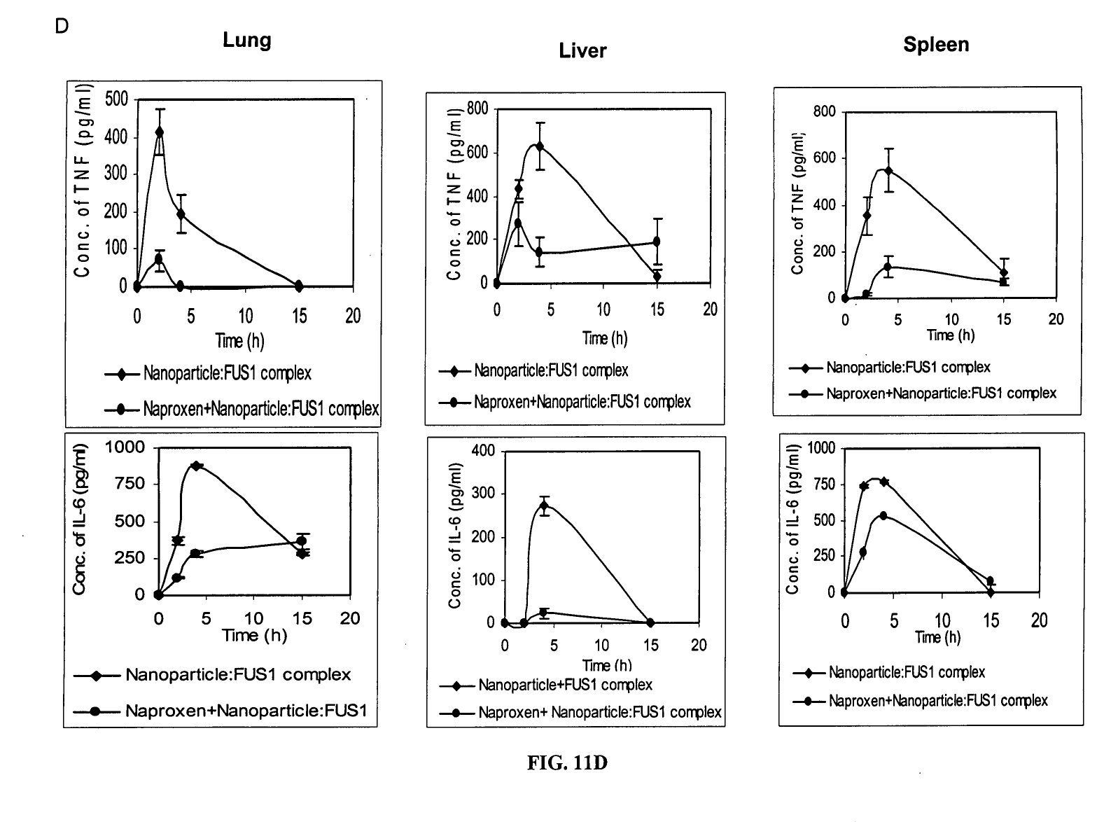

In Vivo Effects of Naproxen on FUS1-nanoparticle induced responses. Mice were intravenously given FUS1-nanoparticle at a dose of 100 μg, which induced 80% lethality within 24 h (FIG. 11). Mice receiving 5% D5W or nanoparticle or FUS1 plasmid alone exhibited no lethality. The effect of naproxen in attenuating the FUS1-nanoparticle induced lung edema and mortality in vivo was then evaluated. Mice were given naproxen (5 or 15 mg/kg, orally) 2 to 3 h before, the treatment of FUS1-nanoparticle (100 μg FUS1). As demonstrated in FIG. 11A, the survival curve shows that the naproxen treatment prior to inject the FUS1-nanoparticle increased the survivorship significantly. Indeed, the percentage of survival was 50% at low concentration drug (5 mg/kg), whereas it was 100% survival at higher concentration of drug treatment (15 mg/kg). Thus naproxen effectively antagonized the FUS1-nanoparticle induced lung toxicity. These results demonstrated that the anti-inflammatory drug, naproxen (15 mg/kg) was effective in inibiting the FUS1-nanoparticle induced mortality. The concentration of naproxen in the plasma was 3.5 μg/200 μl plasma that actually protected mice (FIG. 11B).

Naproxen inhibits FUS1-nanoparticle induced Cytokine activity. Studies were conducted to evaluate the ability of Naproxen to interfere with nanoparticle:FUS1 stimulation of P38, pJNK and p44/42, not only to establish a potential mechanism by which these agents act to inhibit TNF-α, IL-6 biosynthesis but also to determine if MAPK family members are involved in the regulation of TNF-α and IL-6 production. Mice which were intravenously given FUS1-nanoparticle induced a marked increase in serum TNF-α, IL-6, Il-1α and IFN-γ levels, reaching a peak after approximately 2 h, 6 h, 2 h and 6 h (FIG. 11C). As shown in FIG. 11C, pretreatment of mice with Naproxen (15 mg/kg, orally) significantly inhibited serum TNF-α, IL-6, Il-1α and IFN-γ levels induced by FUS1-nanoparticle. Next, studies were conducted to investigate whether naproxen inhibit cytokine secretion induced by FUS1-nanoparticle in the organs. As demonstrated in FIG. 11D, naproxen significantly inhibited TNF-α, IL-6 levels in lungs, liver and spleen.

Naproxen inhibits FUS1-nanoparticle induced MAPK activity. Studies were conducted to test whether naproxen could prevent the phosphorylation of MAPK and its substrates pATF2, pcJun and also phosphorylation of STAT3 in lungs after the administration of FUS1-nanoparticle in vivo. Mice were given naproxen (15 mg/kg) 2 h before, the treatment of FUS1-nanoparticle and lungs, liver and spleen were harvested at different time points. An induction of p38, JNK, ERK pATF2, pcJun phosphorylation and STAT3 phosphorylation at Ser 727 and Tyr 705 at 2 h was observed with persistent activation at 4 h in lung. Pretreatment of mice with naproxen (15 mg/kg) used in this study caused a complete inhibition of p38 at all the time points and partial inhibition of JNK and ERK noticed at 2 and 4 h. Naproxen also inhibited phosphorylation of ATF-2, cJUN and phosphoyrlation of STAT3 at Ser 727 and Tyr705 at 4 h and 15 h. Naproxen treatment also caused inhibition of COX-2 induced by FUS1-nanoparticle at 2 h. Naproxen treatment also inhibited phosphorylation of JNK, ATF-2, cJun, STAT3 ser727 and Tyr 705 in both liver ans spleen. Collectively these results demonstrated that pretreatment of naproxen completely inhibited FUS1-nanoparticle mediated inflammatory cytokine producing MAPK signaling pathway in lungs, liver and spleen.

Effect of Naproxen on FUS1-nanoparticle induced activation of NFκB and STAT3. Studies were conducted to examine the binding capacity of NFκB and STAT3 to DNA in vivo when stimulated with FUS1-nanoparticle complex. Nuclear extract were prepared from whole lung and analyzed by EMSA for transcription factors STAT3 and NFκB. It was found that FUS1-nanoparticle induced significant increase in the NFκB-DNA and STAT3-DNA binding activity at 2 h with decreased activation noticed at 4 h, whereas the binding activity was not seen in control. The activation of STAT3 increased at 2 h with persistent activation at 4 h. To clarify the mechanism of action of the naproxen for the inhibition of the FUS1-nanoparticle induced production of TNF-α, IL-6, effects of naproxen on liposome:FUS1 induced activation of NFκB and STAT3 were examined in lungs. In the presence of naproxen, the activation of NFκB and STAT3 was suppressed. These findings indicate that the inhibition of the FUS1-nanoparticle induced production of TNF-α and IL-6 by naproxen in lungs is induced through the suppression of the FUS1-nanoparticle induced activation of NFκB and STAT3. These results suggest naproxen effectively abolished binding of NFκB and STAT3 to the promoter region of TNF-α, IL-6 and COX-2 genes.

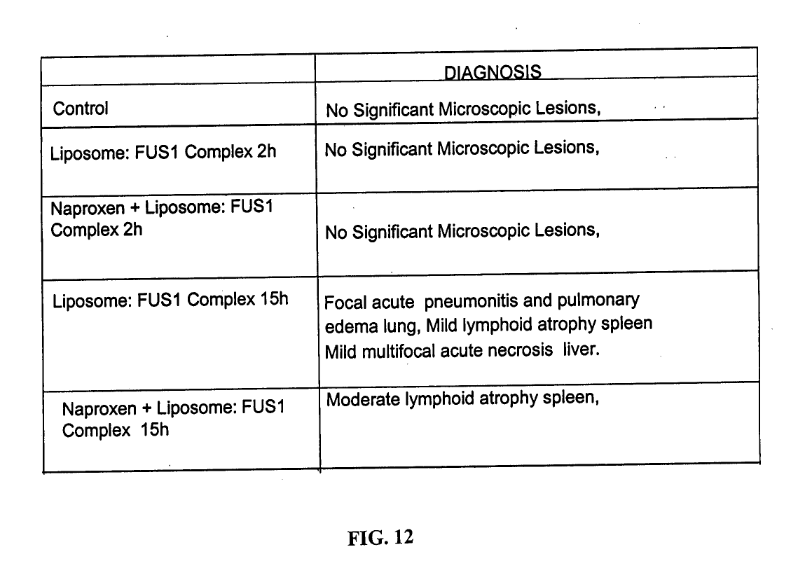

Organ Toxicity: Histology showed that FUS1-nanoparticle has no significant toxicity at 2 h. FUS1-nanoparticle associated toxicity was observed at 15 h. As shown in FIG. 12, the mice developed focal acute pneumonitis, pulmonary edema lung, mild lymphoid atrophy spleen and mild multifocal acute necrosis liver. The naproxen treatment almost completely prevented all the inflammatory features except moderate lymphoid atrophy spleen.

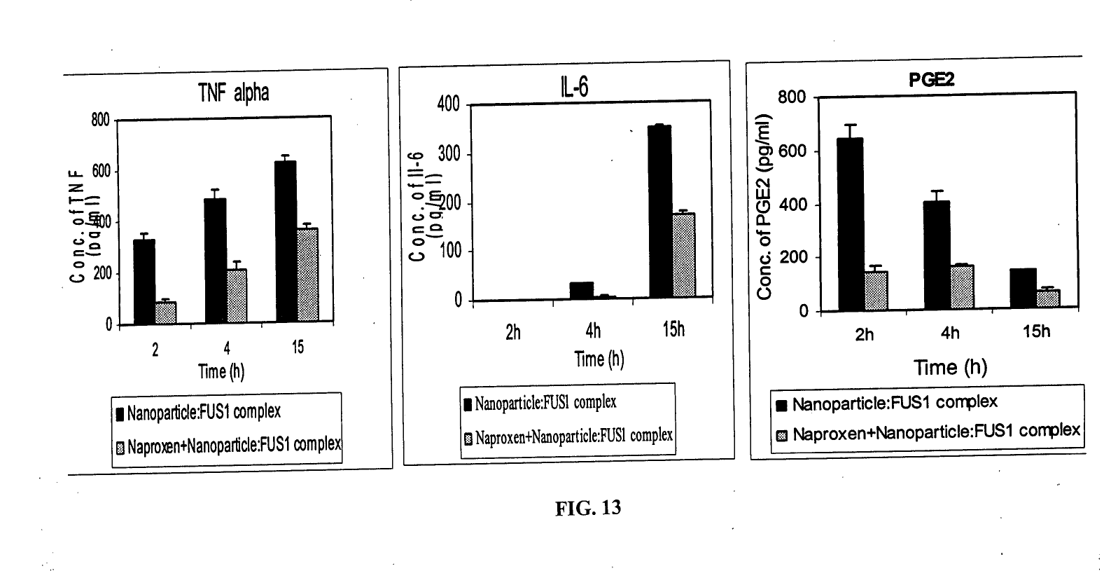

Effects of Naproxen on activation of p38MAPK, JNK/SAPK and p44/42 MAP kinase in vitro. Studies were conducted to investigate whether naproxen could inhibit inflammatory cytokines in RAW 264.7 macrophage cell line. It was found that naproxen suppressed the production of TNF-α, IL-6 and PGE₂ in this cell line. Studies were then conducted to examine the inhibiton of MAP kinases by naproxen. As demonstrated in FIG. 13, FUS1-nanoparticle stimulated a rapid and transient increase within 2 h in the levels of p38MAPK and JNK activities and their substrates ATF2 and cJun, which persisted at 4 h and declined to basal level at 15 h. Studies were then conducted to examine the effects of naproxen on the activation of p38MAPK, JNK and p44/42 MAPK in FUS1-nanoparticle stimulated RAW 264.7 macrophages. Pretreatment of RAW 264.7 macropahge cells with 0.5 mM naproxen significantly inhibited p38MAPK, pJNK, pATF-2 and pcjun at 2 and 4 h. Naproxen also inhibited phosphorylation of STAT 3 at 4 and 15 h. The expression of COX-2 was also inhibited by naproxen at 4 h.

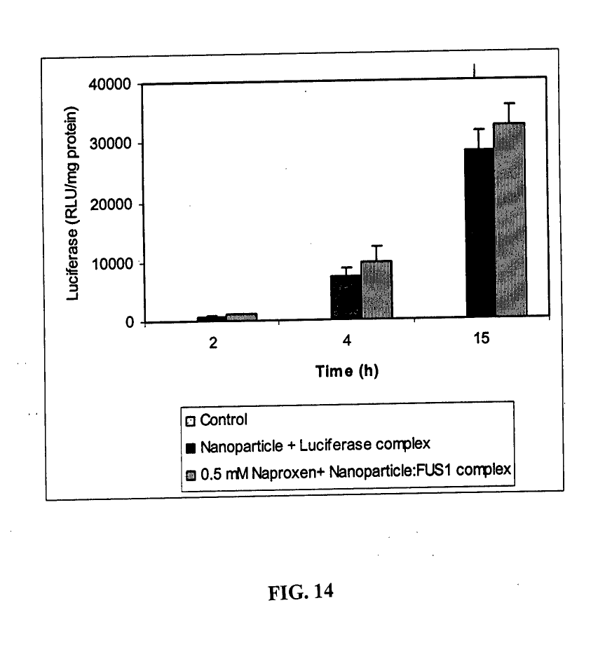

Studies were then conducted to examine whether naproxen affected the transgene expression in RAW 264.7 cells. It was found that pretreatment of cells with 0.5 mM of naproxen did not inhibit transgene (luciferase) expression but rather slightly enhanced the transgene expression over time (FIG. 14). These results suggest that naproxen selectively inhibits the inflammatory response without affecting the transgene expression.

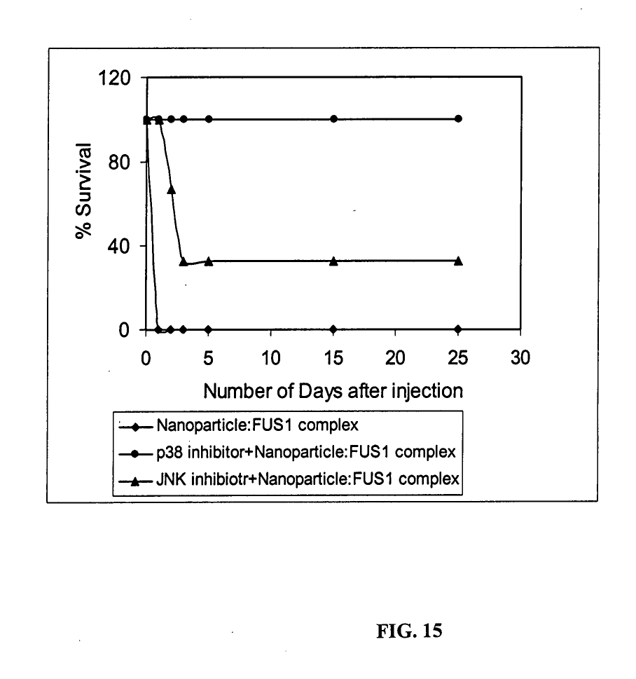

Small molecule inhibitor targeted to p38MAPK and not pJNK protects mice from FUS1-nanoparticle-mediated toxicity. Studies analyzing the inflammatory response induced by FUS1-nanoparticle shoed p38MAPK and pJNK to be activated earlier than p42/44MAPK. To determine if p38MAPK or pJNK initiated the inflammatory cascade and their suppression would protect mice from toxicity, pilot studies were conducted using small molecule inhibitors targeted to p38MAPK and pJNK (FIG. 15). C3H mice were divided into 3 groups: group 1 received FUS1 nanoparticles; Group 2 received p38MAPK inhibitor (SB 203580) intraperitoneally (15 mg/kg) 24 h and 3 h prior to receiving FUS1-nanoparticles; Group 3 received pJNK inhibitor intraperitoneally (15 mg/kg) 24 h and 3 h prior to receiving FUS1 nanoparticles. The amount of FUS1 plasmid DNA delivered was 100 μg. Animals were injected intravenously with FUS1-nanoparticles and animal survival monitored for 25 days. Animals in Group 1 died within 48 h; animals in group 3 treated with pJNK inhibitor showed 33% survival; animals in group 2 treated with p38MAPK inhibitor showed 100% survival. These results indicate that p38MAPK plays a major role in initiating the FUS1-nanoparticle mediated inflammatory response and toxicity. Thus suppression of p38MAPK using small molecule inhibitor provides an alternate strategy to overcome toxicity induced following systemic delivery of FUS1-nanoparticles.

All of the methods and compositions disclosed and claimed herein can be made and executed without undue experimentation in light of the present disclosure. While the compositions and methods of this invention have been described in terms of preferred embodiments, it will be apparent to those of skill in the art that variations may be applied to the methods and compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
U.S. Pat. No. 4,162,282
U.S. Pat. No. 4,310,505
U.S. Pat. No. 4,533,254
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,659,774
U.S. Pat. No. 4,682,195
U.S. Pat. No. 4,683,202
U.S. Pat. No. 4,728,575
U.S. Pat. No. 4,728,578
U.S. Pat. No. 4,737,323
U.S. Pat. No. 4,816,571
U.S. Pat. No. 4,921,706
U.S. Pat. No. 4,959,463
U.S. Pat. No. 5,141,813
U.S. Pat. No. 5,214,136
U.S. Pat. No. 5,223,618
U.S. Pat. No. 5,264,566
U.S. Pat. No. 5,378,825
U.S. Pat. No. 5,401,511
U.S. Pat. No. 5,428,148
U.S. Pat. No. 5,432,260
U.S. Pat. No. 5,446,137
U.S. Pat. No. 5,466,786
U.S. Pat. No. 5,470,967
U.S. Pat. No. 5,539,082
U.S. Pat. No. 5,539,082
U.S. Pat. No. 5,554,744
U.S. Pat. No. 5,574,146
U.S. Pat. No. 5,602,240
U.S. Pat. No. 5,602,244
U.S. Pat. No. 5,603,872
U.S. Pat. No. 5,610,289
U.S. Pat. No. 5,614,617
U.S. Pat. No. 5,623,070
U.S. Pat. No. 5,645,897
U.S. Pat. No. 5,652,099
U.S. Pat. No. 5,670,663
U.S. Pat. No. 5,672,697
U.S. Pat. No. 5,681,947
U.S. Pat. No. 5,700,922
U.S. Pat. No. 5,705,629
U.S. Pat. No. 5,714,331
U.S. Pat. No. 5,714,606
U.S. Pat. No. 5,719,262
U.S. Pat. No. 5,736,336
U.S. Pat. No. 5,763,167
U.S. Pat. No. 5,766,855
U.S. Pat. No. 5,773,571
U.S. Pat. No. 5,777,092
U.S. Pat. No. 5,786,214
U.S. Pat. No. 5,786,461
U.S. Pat. No. 5,792,847
U.S. Pat. No. 5,849,718
U.S. Pat. No. 5,858,988
U.S. Pat. No. 5,859,221
U.S. Pat. No. 5,871,727
U.S. Pat. No. 5,872,232
U.S. Pat. No. 5,879,703
U.S. Pat. No. 5,886,165
U.S. Pat. No. 5,889,155
U.S. Pat. No. 5,891,625
U.S. Pat. No. 5,908,845
U.S. Pat. No. 5,939,277
U.S. Pat. No. 5,994,131
U.S. Pat. No. 6,107,090
U.S. Pat. No. 6,406,921
U.S. Pat. No. 6,457,809
Aksentijevich et al., Hum. Gene Ther., 7(9): 1111-1122, 1996.
Angel et al., Cell, 49: 729, 1987b.
Angel et al., Mol. Cell. Biol., 7: 2256, 1987.
Angel et al., Mol. Cell. Biol., 7: 2256, 1987a.
Atchison and Perry, Cell, 46: 253, 1986.
Atchison and Perry, Cell, 48: 121, 1987.
Ballas et al., J. Immunol., 157: 1840-1845, 1996.
Banerji et al., Cell, 27(2 Pt 1):299-308, 1981.
Banerji et al., Cell, 33(3):729-740, 1983.
Bangham et al., J. Mol. Biol., 13(1):253-259, 1965.
Batttz et al., Biochim. Biophys. Acta., 1535: 100-109, 2001.
Berkhout et al., Cell, 59: 273-282, 1989.
Blanar et al., EMBO J, 8: 1139, 1989.
Bodine and Ley, EMBO J, 6: 2997, 1987.
Boshart et al., Cell, 41: 521, 1985.
Bosze et al, EMBO J., 5(7):1615-1623, 1986.
Braddock et al., Cell, 58: 269, 1989.
Bulla and Siddiqui, J. Virol., 62: 1437, 1986.
Campbell and Villarreal, Mol. Cell. Biol., 8: 1993, 1988.
Campere and Tilghman, Genes and Dev., 3: 537, 1989.
Campo et al., Nature, 303: 77, 1983.
Canfield et al., Methods Enzymol., 189: 418-422, 1990.
Catalysis in Micellar and Macromoleclar Systems, Fendler and Fendler (Eds.), NY, Academic Press, 1975.
Celander and Haseltine, J. Virology, 61: 269, 1987.
Celander et al., J. Virology, 62: 1314, 1988.
Chandler et al., Cell, 33: 489, 1983.
Chang et al., Mol. Cell. Biol., 9: 2153, 1989.
Chatterjee et al., Proc. Natl. Acad. Sci. USA, 86: 9114, 1989.
Cheng et al., Invest. Radiol., 22(1):47-55, 1987.
Choi et al., Cell, 53: 519, 1988.
Chu et al., J. Exp. Med., 186: 1623-1631, 1997.
Cohen et al., J. Cell. Physiol., 5: 75, 1987.
Colloidal Surfactants, Shinoda et al. (Eds.), NY Academic Press, 1963.
Costa et al., Mol. Cell. Biol., 8: 81, 1988.
Cripe et al., EMBO J, 6: 3745, 1987.
Culotta and Hamer, Mol. Cell. Biol., 9: 1376, 1989.
Dandolo et al., J Virology, 47: 55-64, 1983.
De Villiers et al., Nature, 312(5991):242-246, 1984.
Dean et al., J. Chem. Soc., 6655-6659, 1965.
Deschamps et al., Science, 230: 1174-1177, 1985.
Dow et al., J. Immunol., 163: 1552-1561, 1999.
Edbrooke et al., Mol. Cell. Biol., 9: 1908, 1989.
Edlund et al., Science, 230: 912-916, 1985.
El-Gorab et al., Biochim. Biophys. Acta, 306(1):58-66, 1973.
European Pat. Appln. EP 01219
Feigner et al., Proc. Natl. Acad. Sci. USA, 84(21):7413-7417, 1987.
Feng and Holland, Nature, 334: 6178, 1988.
Firak and Subramanian, Mol. Cell. Biol., 6: 3667, 1986.
Fire et al., Nature, 391: 806-811, 1998.
Foecking and Hofstetter, Gene, 45(1):101-105, 1986.
Fraley et al., Proc. Natl. Acad. Sci. USA, 76: 3348-3352, 1979.
Froehler et al., Nucleic Acids Res., 14(13):5399-5407, 1986.
Fujita et al., Cell, 49: 357, 1987.
Gabizon et al., Cancer Res., 50(19):6371-6378, 1990.
Gao et al., J. Immunol. 172: 7703-7712, 2004.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh et al., Infect. Immun., 69: 7703-7710, 2001.
Gilles et al., Cell, 33: 717, 1983.
Gilliland et al., Proc. Natl. Acad. Sci. USA, 77(8):4539-4543, 1980.
Gloss et al., EMBO J., 6: 3735, 1987.
Godbout et al., Mol. Cell. Biol., 8: 1169, 1988.
Goodbourn and Maniatis, Proc. Natl. Acad. Sci. USA, 85: 1447, 1988.
Goodbourn et al., Cell, 45: 601, 1986.
Greene et al., Immunology Today, 10: 272, 1989
Gregoriadis, In: Drug Carriers in Biology and Medicine, Gregoriadis (Ed.), 287-341, 1979.
Grishok et al., Science, 287: 2494-2497, 2000.
Grosschedl and Baltimore, Cell, 41: 885, 1985.
Haslinger and Karin, Proc. Natl. Acad. Sci. USA, 82: 8572, 1985.
Hauber and Cullen, J. Virology, 62: 673, 1988.
Heath et al., Chem. Phys. Lipids, 40: 347, 1986.
Hen et al., Nature, 321: 249, 1986.
Hensel et al., Lymphokine Res., 8: 347, 1989.
Herr and Clarke, Cell, 45: 461, 1986.
Hirochika et al., J. Virol., 61: 2599, 1987.
Hirsch et al., Mol. Cell. Biol., 10: 1959, 1990.
Holbrook et al., Virology, 157: 211, 1987.
Horlick and Benfield, Mol. Cell. Biol., 9: 2396, 1989.
Huang et al., Cell, 27: 245, 1981.
Hug et al., Mol. Cell. Biol., 8: 3065, 1988.
Hwang et al., Mol. Cell. Biol., 10: 585, 1990.
Imagawa et al., Cell, 51: 251, 1987.
Imbra and Karin, Nature, 323: 555, 1986.
Imler et al., Mol. Cell. Biol., 7: 2558, 1987.
Imperiale and Nevins, Mol. Cell. Biol., 4: 875, 1984.
Ito et al., Mol. Ther., 7: 409-418, 2003.
Jakobovits et al., Mol. Cell. Biol., 8: 2555, 1988.
Jameel and Siddiqui, Mol. Cell. Biol., 6: 710, 1986.
Jaynes et al., Mol. Cell. Biol., 8: 62, 1988.
Johnson et al., Mol. Cell. Biol., 9: 3393, 1989.
Kadesch and Berg, Mol. Cell. Biol., 6: 2593, 1986.
Kaneda et al., Science, 243: 375-378, 1989.
Karin et al., Mol. Cell. Biol., 7: 606, 1987.
Karin et al., Mol. Cell. Biol., 7: 606, 1987.
Katinka et al., Cell, 20: 393, 1980.
Kato et al, J. Biol. Chem., 266: 3361-3364, 1991.
Kawarnoto et al., Mol. Cell. Biol., 8: 267, 1988.
Ketting et al., Cell, 99: 133-141, 1999.
Kiledjian et al., Mol. Cell. Biol., 8: 145, 1988.
Kinman et al., Proc. Natl. Acad. Sci. USA, 93: 2879-2883, 1996.
Klamut et al., Mol. Cell. Biol., 10: 193, 1990.
Koch et al., Mol. Cell. Biol., 9: 303, 1989.
Kornberg and Baker, DNA Replication, 2nd Ed., Freeman, San Francisco, 1992.
Krieg et al., Nature, 374: 546-549, 1995.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3: 325, 1983.
Kriegler et al., Cell, 38: 483, 1984.
Kriegler et al., Cell, 53: 45, 1988.
Kuhl et al., Cell, 50: 1057, 1987.
Kumar et al., Nat. Rev. Drug Dis. 2: 717-726, 2003.
Kunz et al., Nucl. Acids Res., 17: 1121, 1989.
Lai et al., J. Immunol. 170: 6244-6249, 2003.
Larsen et al., Proc Natl. Acad. Sci. USA., 83: 8283, 1986.
Laspia et al., Cell, 59: 283, 1989.
Latimer et al., Mol. Cell. Biol., 10: 760, 1990.
Lee et al., Nature, 294: 228, 1981.
Lee et al., Nucleic Acids Res., 12: 4191-206, 1984.
Levinson et al., Nature, 295: 79, 1982.
Li et al., Immunity, 10: 577-584, 1999.
Lin and Avery, Nature, 402: 128-129, 1999.
Lin et al., Mol. Cell. Biol., 10: 850, 1990.
Lipford et al., Eur. J. Immunol., 27: 3420-3426, 1997.
Liposome Technology, Gregoriadis (Ed.), Boca Raton, Fla., 1984.
Liu et al., J. Biol. Chem., 270: 24864-24870, 1995.
Luria et al., EMBO J., 6: 3307, 1987.
Lusky and Botchan, Proc. Natl. Acad. Sci. USA, 83: 3609, 1986.
Lusky et al., Mol. Cell. Biol., 3: 1108, 1983.
Macejak and Sarnow, Nature, 353: 90-94, 1991.
Majors and Varmus, Proc. Natl. Acad. Sci. USA, 80: 5866, 1983.
Manning and Davis, Nat. Rev. Drug Dis. 2: 554-565, 2003.
Martin et al., Nature, 345(6277):739-743, 1990.
Mayer et al., Biochim Biophys Acta, 858(1):161-8, 1986.
Mayer et al., Biochim. Biophys. Acta, 817(1):193-196, 1985.
Mayer et al., Biochim. Biophys. Acta, 858(1):161-168, 1986.

Mayhew et al., Biochim. Biophys. Acta, 775(2):169-174, 1984.
Mayhew et al., Methods Enzymol., 149: 64-77, 1987.
McNeall et al., Gene, 76: 81, 1989.
Miksicek et al., Cell, 46: 203, 1986.
Montaldo et al., Int. J. Cancer, 81(2):262-267, 1999.
Montgomery et al., Proc. Natl. Acad. Sci. USA, 95: 155-2-15507, 1998.
Mordacq and Linzer, Genes and Dev., 3: 760, 1989.
Moreau et al., Nucl. Acids Res., 9: 6047, 1981.
Muesing et al., Cell, 48: 691, 1987.
Mujoo, Maneval, Anderson, Gutterman, Oncogene, 12: 1617-1623, 1996.
Ng et al., Nuc. Acids Res., 17: 601, 1989.
Nicolau and Sene, Biochim. Biophys. Acta, 721: 185-190, 1982.
Nicolau et al., Methods Enzymol., 149: 157-176, 1987.
Ondek et al., EMBO J., 6: 1017, 1987.
Omitz et al., Mol. Cell. Biol., 7: 3466, 1987.
Pagnan et al., Int. J. Cancer, 81(2):268-274, 1999.
Palladino et al., Nat. Rev. Drug Dis., 2: 736-746, 2003.
Palmiter et al., Nature, 300: 611, 1982.
PCT Appln. PCT/US85/01161
PCT Appln. PCT/US89/05040
PCT Appln. WO 84/03564
PCT Appln. WO 92/20702
PCT Appln. WO 98/0748
PCT Appln. WO 99/18933
Pech et al., Mol. Cell. Biol., 9: 396, 1989.
Pelletier and Sonenberg, Nature, 334: 320-325, 1988.
Perez-Stable and Constantini, Mol. Cell. Biol., 10: 1116, 1990.
Philip et al., J. Biol. Chem., 268(22):16087-16090, 1993.
Picard and Schaffner, Nature, 307: 83, 1984.
Pinkert et al., Genes and Dev., 1: 268, 1987.
Ponta et al., Proc. Natl. Acad. Sci. USA, 82: 1020, 1985.
Porton et al., Mol. Cell. Biol., 10: 1076, 1990.
Queen and Baltimore, Cell, 35: 741, 1983.
Quinn et al., Mol. Cell. Biol., 9: 4713, 1989.
Ramesh et al., Biochem. Biophys. Acta., 1083: 121-134, 1991.
Ramesh et al., Mol. Ther., 3: 337-350, 2001a.
Ramesh et al., DNA and Cell Biol., In press, 2001b.
Redondo et al., Science, 247: 1225, 1990.
Reisman and Rotter, Mol. Cell. Biol., 9: 3571, 1989.
Remington's Pharmaceutical Sciences, 15^{th} ed., pages 1035-1038 and 1570-1580, Mack Publishing Company, Easton, Pa., 1980.
Resendez Jr. et al., Mol. Cell. Biol., 8: 4579, 1988.
Ripe et al., Mol. Cell. Biol., 9: 2224, 1989.
Rittling et al., Nuc. Acids Res., 17: 1619, 1989.
Roman et al., Nat. Med., 3: 849-854, 1997.
Rosen et al., Cell, 41: 813, 1988.
Sakai et al., Genes and Dev., 2: 1144, 1988.
Sakurai et al., Gene Therapy 9: 1120-1126, 2002.
Sambrook, In: Molecular Cloning, Cold Spring Harbor Laboratory Press, 2001.
Satake et al., J Virology, 62: 970, 1988.
Schaffner et al., J. Mol. Biol., 201: 81, 1988.
Schulz et al., Cancer Res. 44: 5914-5920, 1984.
Searle et al., Mol. Cell. Biol., 5: 1480, 1985.
Sharp and Marciniak, Cell, 59: 229, 1989.
Sharp and Zamore, Science, 287: 2431-2433, 2000.
Sharp, Genes Dev., 13: 139-141, 1999.
Shaul and Ben-Levy, EMBO J., 6: 1913, 1987.
Sherman et al., Mol. Cell. Biol., 9: 50, 1989.
Sleigh and Lockett, J. EMBO, 4: 3831, 1985.
Solodin et al., Biochemistry, 34(41):13537-13544, 1995.
Spalholz et al., Cell, 42: 183, 1985.
Spandau and Lee, J. Virology, 62: 427, 1988.
Spandidos and Wilkie, EMBO J., 2: 1193, 1983.
Spanjer and Scherphof, Biochim. Biophys. Acta, 21: 734(1):40-47, 1983.
Sparwasser et al., Eur. J. Immunol., 28: 2045-2054, 1998.
Sparwasser et al., Nature, 386: 336-337, 1997.
Stacey et al., J. Immunol., 157: 2116-2122, 1996.
Stephens and Hentschel, Biochem. J, 248: 1, 1987.
Stuart et al., Nature, 317: 828, 1985.
Sullivan and Peterlin, Mol. Cell. Biol., 7: 3315, 1987.
Swartzendruber and Lehman, J. Cell. Physiology, 85: 179, 1975.
Szoka and Papahadjopoulos, Proc. Natl. Acad. Sci. USA, 75: 4194-4198, 1978.
Tabara et al., Cell, 99: 123-132, 1999.
Takebe et al., Mol. Cell. Biol., 8: 466, 1988.
Tan et al., Mol. Cell, 9: 993-1004, 2002.
Tavernier et al., Nature, 301: 634, 1983.
Taylor and Kingston, Mol. Cell. Biol., 10: 165, 1990a.
Taylor and Kingston, Mol. Cell. Biol., 10: 176, 1990b.
Taylor et al., J. Biol. Chem., 264: 15160, 1989.
Templeton et al., Nat. Biotechnol., 15: 647-652, 1997.
Thierry et al., Proc. Natl. Acad. Sci. USA, 92(21):9742-9746, 1995.
Thiesen et al., J Virology, 62: 614, 1988.
Tousignant et al., Hum. Gene Ther., 11: 2493-2513, 2000.
Treisman, Cell, 42: 889, 1985.
Tronche et al., Mol. Biol. Med., 7: 173, 1990.
Trudel and Constantini, Genes and Dev. 6: 954, 1987.
Tsukamoto et al., Nat. Genet., 9(3):243-248, 1995.
Tyndell et al., Nuc. Acids. Res., 9: 6231, 1981.
UK Patent Appln. GB 2193095A
Vannice and Levinson, J. Virology, 62: 1305, 1988.
Vasseur et al., Proc Natl. Acad. Sci. U.S.A., 77: 1068, 1980.
Wagner et al., Science, 260: 1510-1513, 1993.
Wang and Calame, Cell, 47: 241, 1986.
Weber et al., Cell, 36: 983, 1984.
Weinberger et al. Mol. Cell. Biol., 8: 988, 1984.
Winoto and Baltimore, Cell 59: 649, 1989.
Wong et al., Gene, 10: 87-94, 1980.
Yang and Huang, Gene Therapy, 4 (9):950-960, 1997.
Yeo et al., J. Biol. Chem., 278: 22563-22573, 2003. Yutzey et al. Mol. Cell. Biol., 9: 1397, 1989.
Zhu et al., Science, 261(5118):209-211, 1993.

1. A method to prevent or reduce inflammation secondary to administration of a lipid-nucleic acid complex in a subject, comprising administering to the subject an agent with the lipid-nucleic acid complex, wherein the agent is selected from the group consisting of a non-steroidal anti-inflammatory agent, a salicylate, an anti-rheumatic agent, an antihistamine, and an immunosuppressive agent.

2. The method of claim 1, wherein the nucleic acid contains CpG sites that induce inflammation.

3. The method of claim 1, wherein the inflammation is secondary to upregulation of NFκB in the subject.

4. The method of claim 1, wherein the agent is administered to the subject concurrently with the lipid-nucleic acid complex.

5. The method of claim 4, wherein the agent is incorporated into the lipid-nucleic acid complex.

6. The method of claim 1, wherein the agent is administered to the subject separately from the lipid-nucleic acid complex.

7. The method of claim 1, wherein the agent is administered to the subject prior to administration of the lipid-nucleic acid complex.

8. The method of claim 1, wherein the agent is administered to the subject following administration of the lipid-nucleic acid complex.

9. The method of claim 1, further comprising administering to the subject two or more agents selected from the group consisting of a non-steroidal anti-inflammatory agent, a salicylate, an anti-rheumatic agent, an antihistamine, and an immunosuppressive agent.

10. The method of claim 9, further comprising administering to the subject a non-steroidal anti-inflammatory agent and a salicylate, a salicylate and an antirheumatic agent, an antirheumatic agent and an immunosuppressive agent, a non-steroidal antiinflammatory agent and an immunosuppressive agent, a salicylate and an immunosuppressive agent, a non-steroidal anti-inflammatory agent and an antihistamine, a salicylate and an antihistamine, an anti-rheumatic agent and an antihistamine, an immunosuppressive agent and an antihistamine, or a non-steroidal anti-inflammatory agent and an anti-rheumatic agent.

11. The method of claim 1, wherein the non-steroidal anti-inflammatory agent is diflunisal, ibuprofen, fenoprofen, flurbiprofen, ketoprofen, nabumetone, piroxicam, naproxen, naproxen sodium, diclofenac, diclofenac sodium and misoprostol, indomethacin, sulindac, etodolac, tolmetin, etodolac, ketorolac, oxaprozin, rofecoxib, mefenamic acid, meclofenamate, celecoxib, or vioxx.

12. The method of claim 1, wherein the non-steroidal anti-inflammatory agent is naproxen.

13. The method of claim 1, wherein the non-steroidal anti-inflammatory agent is an inhibitor of an inflammation-associated signaling molecule.

14. The method of claim 13, wherein the inflammation-associated signaling molecule is p38MAPK or p44/42MAPK.

15. The method of claim 14, wherein the inhibitor of p38MAPK is SB 203580.

16. The method of claim 14, wherein the inhibitor of p44/42MAPK is U0126.

17. The method of claim 1, wherein the salicylate is acetylsalicylic acid, sodium salicylate, choline salicylate, choline magnesium salicylate, diflunisal, salsalate, or choline magnesium trisalicylate.

18. The method of claim 1, wherein the anti-rheumatic agent is gold sodium thiomalate, aurotheioglucose, auranofin, chloroquine, hydroxychloroquine, penicillamine, leflunomide, etanercept, infliximab, azathioprine, or sulfasalazine.

19. The method of claim 1, wherein the antihistamine is diphenhydramine, chlorpheniramine, clemastine, hydroxyzine, triprolidine, loratadine, cetirizine, fexofenadine, or desloratadine.

20. The method of claim 1, wherein the immunosuppressive agent is cyclosporine A, azathoprine, methotrexate, mechorethamine, cyclophosphamide, chlorambucil, or mycophenolate mofetil.

21. The method of claim 20, wherein the immunosuppressive agent is cyclosporine A.

22. The method of claim 1, wherein the nucleic acid is a deoxyribonucleic acid (DNA).

23. The method of claim 22, wherein the deoxyribonucleic acid is a therapeutic gene.

24. The method of claim 23, wherein the therapeutic gene is a tumor suppressor gene, a gene that induces apoptosis, a gene encoding an enzyme, a gene encoding an antibody, or a gene encoding a hormone.

25. The method of claim 24, wherein the therapeutic gene is Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, FUS1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, Gene 26 (CACNA2D2), PL6, Beta*(BLU), LUCA-1 (HYAL1), LUCA-2 (HYAL2), 123F2 (RASSF1), 101F6, Gene 21 (NPRL2), SEM A3 or MCC.

26. The method of claim 25, wherein the therapeutic gene is fus-1.

27. The method of claim 1, wherein the nucleic acid is antisense ras, antisense myc, antisense raf, antisense erb, antisense src, antisense fms, antisense jun, antisense trk, antisense ret, antisense gsp, antisense hst, antisense bcl, or antisense abl.

28. The method of claim 1, wherein the nucleic acid is ribonucleic acid (RNA).

29. The method of claim 28, wherein the RNA is messenger RNA, antisense RNA, interfering RNA, or RNA comprised in a ribozyme.

30. The method of claim 1, wherein the nucleic acid is a DNA-RNA hybrid.

31. The method of claim 1, wherein the lipid is a cationic lipid.

32. The method of claim 1, wherein the cationic lipid is DOTAP or DOTMA.

33. The method of claim 1, wherein the lipid is a neutral lipid.

34. The method of claim 33, wherein the neutral lipid is DOPE.

35. The method of claim 1, wherein the lipid further comprises a liposome.

36. The method of claim 35, wherein the liposome is a unilamellar liposome or a multilamellar liposome.

37. The method of claim 1, wherein the lipid is comprised in a nanoparticle.

38. The method of claim 1, wherein the lipid-nucleic acid complex comprises a composition that includes DOTAP, cholesterol, and FUS1, and wherein the non-steroidal anti-inflammatory agent is naproxen.

39. The method of claim 1, wherein the lipid-nucleic acid complex comprises a composition that includes DOTAP, cholesterol, and FUS1, and wherein the non-steroidal anti-inflammatory agent is cyclosporine A.

40. A method of screening for inhibitors of the inflammatory response associated with administration of a lipid-nucleic acid complex to a subject, comprising:
(a) providing a candidate substance suspected of preventing or inhibiting the inflammation associated with administration of a lipid-nucleic acid complex;
(b) contacting a composition comprising the lipid-nucleic acid complex and the candidate substance with the subject, and
(c) assaying for inflammation in the subject.

41. A composition comprising:
(a) a lipid;
(b) a nucleic acid; and
(c) a non-steroidal anti-inflammatory agent, a salicylate, an anti-rheumatic agent, an antihistamine, or an immunosuppressive agent.
